(19) United States
(12) Patent Application Publication
(10) Pub. No.: US 2025/0244342 A1
(43) Pub. Date: Jul. 31, 2025

(54) METHODS AND KITS FOR ASSESSING ALZHEIMER'S DISEASE

(71) Applicants: MESO SCALE TECHNOLOGIES, LLC., Rockville, MD (US); THE GENERAL HOSPITAL CORPORATION, Boston, MA (US)

(72) Inventors: Martin STENGELIN, Gaithersburg, MD (US); Christopher CAMPBELL, Columbia, MD (US); Steven E. ARNOLD, Charlestown, MA (US); Pia KIVISÄKK WEBB, Charlestown, MA (US)

(21) Appl. No.: 18/854,225

(22) PCT Filed: Apr. 6, 2023
(86) PCT No.: PCT/US2023/065471

§ 371 Date: Oct. 4, 2024

Related U.S. Application Data
(60) Provisional application No. 63/373,951, filed on Aug. 30, 2022.
(60) Provisional application No. 63/362,641, filed on Apr. 7, 2022.

(51) Int. Cl. G01N 33/68; C12Q 1/6844; G01N 21/76

(52) U.S. Cl. CPC G01N 33/6896; C12Q 1/6844; G01N 21/76; G01N 2800/2814; G01N 2800/2821; G01N 2800/50; G01N 2800/52

(57) ABSTRACT

The disclosure relates to methods and kits for detecting tau, e.g., tau that is phosphorylated at amino acid position T181 (pTau181), tau that is phosphorylated at amino acid position T217 (pTau217), and/or total tau. The disclosure further provides methods for distinguishing between individuals whose cognitive condition will remain stable and whose cognitive condition will decline during their lifetime. The disclosure also provides methods for determining the eligibility of individuals for participation in clinical trials for Alzheimer's disease treatments. Also provided are methods for distinguishing between individuals with Alzheimer's disease and non-Alzheimer's dementia, and for monitoring response to treatment for Alzheimer's disease.

### GOVERNMENT LICENSE RIGHTS

This invention was made with government support under contract AI118663 awarded by the National Institutes of Health. The government has certain rights in the invention.

### FIELD OF THE INVENTION

The disclosure relates to methods and kits for detecting tau, e.g., tau that is phosphorylated at amino acid position T181 (pTau181), tau that is phosphorylated at amino acid position T217 (pTau217), and/or total tau. The disclosure further provides methods for distinguishing between individuals whose cognitive condition will remain stable and whose cognitive condition will decline during their lifetime. The disclosure also provides methods for determining the eligibility of individuals for participation in clinical trials for Alzheimer's disease treatments. Also provided are methods for distinguishing between individuals with Alzheimer's disease and non-Alzheimer's dementia, and for monitoring response to treatment for Alzheimer's disease.

### BACKGROUND

Diagnosis of early stage Alzheimer's pathology is valuable for early and more effective treatment and for enrolling patients into clinical trials. Further, early stage diagnosis is important for identifying individuals for whom treatment would likely provide the greatest benefit. Currently, Alzheimer's disease pathology can be diagnosed using invasive and expensive methods such as PET imaging and spinal tap to obtain cerebrospinal fluid (CSF). In embodiments, the present disclosure provides highly sensitive, specific, and non-invasive methods of assessing Alzheimer's disease.

### SUMMARY OF THE INVENTION

In embodiments, the disclosure provides a method of detecting phosphorylated tau (pTau) in a sample, wherein the pTau is phosphorylated at amino acid position T181 (pTau181) or amino acid position T217 (pTau217), comprising: (a) contacting the sample with: (i) a first capture reagent that binds tau; (ii) a second capture reagent that binds tau; (iii) a detection reagent that binds pTau181 or pTau217, thereby forming a complex comprising the first and second capture reagents, pTau181 or pTau217, and the detection reagent; and (b) detecting the complex, thereby detecting the pTau181 or pTau217. In embodiments, the sample is from a subject diagnosed with mild cognitive impairment (MCI) or at risk of developing MCI. In embodiments, the sample is from a subject with subjective cognitive complaints (SCC). In embodiments, the sample comprises less than 75% pTau181 as compared to a normalized pTau181 concentration, and/or wherein the sample comprises less than 75% pTau217 as compared to a normalized pTau217 concentration. In embodiments, the sample comprises greater than 120% pTau181 as compared to a normalized pTau181 concentration, and/or wherein the sample comprises greater than 120% pTau217 as compared to a normalized pTau217 concentration. In embodiments, the complex is bound to a surface prior to the detecting.

In embodiments, the disclosure provides a method of detecting pTau181 or pTau217 in a sample, comprising: (a) contacting the sample with: (i) a first capture reagent that binds tau; (ii) a second capture reagent that binds tau; (iii) a first detection reagent that binds tau; and (iv) a second detection reagent that binds pTau181 or pTau271, thereby forming a complex comprising the first and second capture reagents, pTau181 or pTau217, and the first and second detection reagents; and (b) detecting the complex, thereby detecting the pTau181 or pTau217. In embodiments, the sample is from a subject diagnosed with mild cognitive impairment (MCI) or at risk of developing MCI. In embodiments, the sample is from a subject with subjective cognitive complaints (SCC). In embodiments, the sample comprises less than 75% pTau181 as compared to a normalized pTau181 concentration, and/or wherein the sample comprises less than 75% pTau217 as compared to a normalized pTau217 concentration. In embodiments, the sample comprises greater than 120% pTau181 as compared to a normalized pTau181 concentration, and/or wherein the sample comprises greater than 120% pTau217 as compared to a normalized pTau217 concentration. In embodiments, the complex is bound to a surface prior to the detecting.

In embodiments, the disclosure provides a method of detecting phosphorylated tau (pTau) in a sample, wherein the pTau is phosphorylated at amino acid position T217 (pTau217), comprising: (a) contacting the sample with: (i) a capture reagent that specifically binds pTau217; and (ii) a detection reagent that binds tau, thereby forming a complex comprising the capture reagent, pTau217, and the detection reagent; and (b) detecting the complex, thereby detecting the pTau217.

In embodiments, the disclosure provides a method of detecting phosphorylated tau (pTau) in a sample, wherein the pTau is phosphorylated at amino acid position T181 (pTau181), comprising: (a) contacting the sample with: (i) a capture reagent that specifically binds pTau181; and (ii) a detection reagent that binds tau, thereby forming a complex comprising the capture reagent, pTau181, and the detection reagent; and (b) detecting the complex, thereby detecting the pTau181.

In embodiments, the disclosure provides a method of detecting phosphorylated tau (pTau) in a sample, wherein the pTau is phosphorylated at amino acid position T181 (pTau181), comprising: (a) contacting the sample with: (i) a capture reagent that specifically binds pTau181; (ii) a first detection reagent that binds tau; and (iii) a second detection reagent that binds tau, thereby forming a complex comprising the capture reagent, pTau181, and the detection reagents; and (b) detecting the complex, thereby detecting the pTau181.

In embodiments, the disclosure provides a method of determining if a cognitively normal subject is likely to experience cognitive decline within 10 years, comprising: (a) measuring pTau181, pTau217, and/or total tau levels in a sample obtained from the subject; and (b) determining if the subject is likely to experience cognitive decline based on the pTau181, pTau217, and/or total tau levels measured in (a). In embodiments, the disclosure provides a method of preventing, reducing, or delaying cognitive decline in a cognitively normal subject, comprising: (a) measuring pTau181, pTau217, and/or total tau levels in a sample obtained from the subject; (b) identifying the subject as being likely to experience future cognitive decline, e.g., within 10 years, based on the pTau181, pTau217, and/or total tau levels measured in (a); and (c) administering a regimen to the subject to prevent, reduce, or delay cognitive decline.

In embodiments, the disclosure provides a method of determining if a subject with SCC is likely to experience cognitive decline within 10 years, comprising: (a) measuring pTau181, pTau217, and/or total tau levels in a sample obtained from the subject; and (b) determining if the subject is likely to experience cognitive decline based on the pTau181, pTau217, and/or total tau levels measured in (a). In embodiments, the disclosure provides a method of preventing, reducing, or delaying cognitive decline in a subject with subjective cognitive complaints, comprising: (a) measuring pTau181, pTau217, and/or total tau levels in a sample obtained from the subject; (b) identifying the subject as being likely to experience future cognitive decline, e.g., within 10 years, based on the pTau181, pTau217, and/or total tau levels measured in (a); and (c) administering a regimen to the subject to prevent, reduce, or delay cognitive decline.

In embodiments, the disclosure provides a method of determining if a subject with MCI is likely to experience further cognitive decline within 10 years, comprising: (a) measuring pTau181, pTau217, and/or total tau levels in a sample obtained from the subject; and (b) determining if the subject is likely to experience further cognitive decline based on the pTau181, pTau217, and/or total tau levels measured in (a). In embodiments, the disclosure provides a method of preventing, reducing, or delaying further cognitive decline in a subject with MCI, comprising: (a) measuring pTau181, pTau217, and/or total tau levels in a sample obtained from the subject; (b) identifying the subject as being likely to experience further cognitive decline, e.g., within 10 years, based on the pTau181, pTau217, and/or total tau levels measured in (a); and (c) administering a regimen to the subject to prevent, reduce, or delay further cognitive decline.

In embodiments, the disclosure provides a method of distinguishing a subject afflicted with AD from an individual afflicted with non-Alzheimer's dementia, the method comprising: (a) measuring pTau181, pTau217, and/or total tau levels in a sample obtained from the subject; and (b) identifying, based on the pTau181, pTau217, and/or total tau levels measured in (a), the subject as (i) afflicted with AD or (ii) afflicted with non-Alzheimer's dementia. In embodiments, the disclosure provides a method of treating AD in a subject in need thereof, comprising: (a) measuring pTau181, pTau217, and/or total tau levels in a sample obtained from the subject, wherein the sample is obtained prior to administration of a treatment for AD; (b) determining, based on the pTau181, pTau217, and/or total tau levels measured in (a), that the subject is afflicted with Alzheimer's disease; and (c) administering a treatment regimen for AD to the subject.

In embodiments, the disclosure provides a method of monitoring response to treatment for AD in a subject, comprising: (a) measuring pTau181, pTau217, and/or total tau levels in a sample obtained from the subject, wherein the sample is obtained prior to administration of a treatment regimen for AD; (b) measuring pTau181, pTau217, and/or total tau levels in a sample obtained from the individual at one or more time points after administration of the treatment regimen for AD has been initiated; (c) determining, based on the pTau181, pTau217, and/or total tau levels measured in (a) and (b), that the subject is responding positively to the treatment regimen; and (d) continuing to administer the treatment regimen for AD to the subject.

In embodiments, the disclosure provides a method of determining eligibility of a subject to participate in a clinical trial of a therapeutic drug for preventing or delaying AD, the method comprising: (a) measuring pTau181, pTau217, and/or total tau levels in a sample obtained from the subject; (b) determining the eligibility of the subject for the clinical trial based on the pTau181, pTau217, and/or total tau levels measured in (a). In embodiments, the subject has been diagnosed with dementia or MCI. In embodiments, the subject has SCC. In embodiments, the subject does not have any cognitive impairment. In embodiments, the disclosure provides a method of conducting a clinical trial of a therapeutic drug or intervention for Alzheimer's disease, the method comprising: (a) measuring pTau181, pTau217, and/or total tau levels in a sample obtained from a subject; (b) determining eligibility of the subject for the clinical trial based on the pTau181, pTau217, and/or total tau levels measured in (a); and (c) administering the therapeutic drug to the subject.

In embodiments, the disclosure provides a kit for detecting pTau181 and/or pTau217 comprising, in one or more vials, containers, or compartments: (a) a first capture reagent that binds tau; (b) a second capture reagent that binds tau; (b) a detection reagent that binds pTau181 and/or a detection reagent that binds pTau217; and (d) optionally a surface, wherein the first and second capture reagents are provided on the surface or are capable of binding to the surface.

In embodiments, the disclosure provides a kit for detecting pTau181 comprising, in one or more vials, containers, or compartments: (a) a first capture reagent that binds tau; (b) a second capture reagent that binds tau; (c) a first detection reagent that binds tau; (d) a second detection reagent that binds pTau181; and (e) optionally a surface, wherein the first and second capture reagents are provided on the surface or are capable of binding to the surface.

In embodiments, the disclosure provides a kit for detecting pTau181 comprising, in one or more vials, containers, or compartments: (a) a capture reagent that binds pTau181; (b) a detection reagent that binds tau; and (c) optionally a surface, wherein the capture reagent is provided on the surface or is capable of binding to the surface.

In embodiments, the disclosure provides a kit for detecting pTau181 comprising, in one or more vials, containers, or compartments: (a) a capture reagent that binds pTau181; (b) a first detection reagent that binds tau; (c) a second detection reagent that binds tau; and (d) optionally a surface, wherein the capture reagent is provided on the surface or is capable of binding to the surface.

In embodiments, the disclosure provides a kit for detecting pTau217 comprising, in one or more vials, containers, or compartments: (a) a capture reagent that binds pTau217; (b) a detection reagent that binds tau; and (c) optionally a surface, wherein the capture reagent is provided on the surface or are capable of binding to the surface.

### BRIEF DESCRIPTION OF THE DRAWINGS

The following drawings form part of the present specification and are included to further demonstrate exemplary embodiments of certain aspects of the present invention.

FIGS. 1A and 1B show the results of assays to measure pTau181 levels in individuals categorized under various diagnostic groups as described in embodiments herein. In FIG. 1A, pTau181 levels were measured with an assay using first and second capture reagents (“dual-capture assay”) and a detection reagent linked to a nucleic acid probe, as described in embodiments herein (“pTau181 Assay Format 1”). In FIG. 1B, pTau181 levels were measured using a dual-capture assay with a first detection reagent linked to a first nucleic acid probe, and a second detection reagent linked to a second nucleic acid probe, as described in embodiments herein (“pTau181 Assay Format 2”).

FIGS. 2A and 2B show the results of assays to measure pTau181 levels in individuals categorized under various diagnostic groups according to CDR® Dementia Staging Instrument scores as described in embodiments herein. In FIG. 2A, pTau181 levels were measured using pTau181 Assay Format 1. In FIG. 2B, pTau181 levels were measured using pTau181 Assay Format 2.

FIGS. 3A and 3B show receiver operating characteristic (ROC) curves for using pTau181 to distinguish between individuals in the Normal (high contrast) group versus the AD, AD (Path), and AD (high contrast) groups (collectively “AD groups”). In FIG. 3A, pTau181 levels were measured using pTau181 Assay Format 1. In FIG. 3B, pTau181 levels were measured using pTau181 Assay Format 2.

FIG. 4 shows an ROC curve for using total tau to distinguish between individuals in the Normal group and AD groups. Total tau levels were measured using a dual-capture assay with a detection reagent comprising a detectable label, as described in embodiments herein (“total tau Assay Format 2”).

FIG. 5 shows an ROC curve for using pTau181 to distinguish between individuals in the Normal group and the following groups, which are indicative of early disease: Normal, decline; SCC, stable; subjective cognitive complaints (SCC), decline; mild cognitive impairment (MCI), stable; MCI, decline; MCI, AD; and MCI, Non-AD. Levels of pTau181 were measured using pTau181 Assay Format 2.

FIG. 6 shows an ROC curve for using pTau181 to distinguish between individuals in the Normal, stable group (i.e., cognitively normal individuals who remain stable) and the Normal, decline group (i.e., cognitively normal individuals who later decline). Levels of pTau181 were measured using pTau181 Assay Format 1.

FIG. 7 shows an ROC curve for using pTau181 to distinguish between individuals in the MCI, stable group (i.e., individuals with MCI who remain stable) and the MCI, decline group (i.e., individuals with MCI who later decline). Levels of pTau181 were measured using pTau181 Assay Format 2.

FIG. 8A shows the plasma levels of pTau181, and FIG. 8B shows the plasma levels of total tau (natural logarithm (In) of measured levels shown), measured in individuals with AD, other neurodegenerative diseases (OND), and healthy controls (HC). Box plots show the median and 25^{th}/75^{th} percentile.

FIG. 9A shows the plasma levels of pTau181, and FIG. 9B shows the plasma levels of total tau (natural logarithm (In) of measured levels shown), measured in individuals with AD, MCI, SCC, or no cognitive impairment (CU), stratified by the presence (decline) or absence (stable) of cognitive decline during 4 years of follow-up. Box plots show the median and 25^{th}/75^{th} percentile.

FIG. 10 shows the plasma levels of pTau181 and total tau in HC individuals, individuals with AD, and individuals with OND as described in embodiments herein; fold change in biomarker levels between groups, and AUC values for differentiation between groups.

FIG. 11 shows the plasma levels of pTau181 and total tau in MCI-decline and MCI-stable individuals as described in embodiments herein; fold change in biomarker levels between groups; and AUC values for differentiation between groups.

FIGS. 12A-12D show the results of a pTau217 assay as described in embodiments herein, for measuring plasma pTau217 levels in AD vs. control groups as described in Example 4. FIGS. 12A shows the measured pTau217 levels in the AD vs. control groups. FIG. 12B shows the pTau217 concentrations by diagnosis in the AD vs. control groups. FIG. 12C shows the pTau217 levels grouped by disease stage. FIG. 12D shows the AUC curve of the pTau217 assay.

FIG. 13A shows the correlation of pTau217 levels with age, as described in Example 4. FIG. 13B shows the correlation of the levels of pTau217 and pTau181 as measured by assays described in embodiments herein.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y and any numbers that fall within x and y.

As used herein, an individual who is “cognitively normal” or “cognitively healthy” refers to a person who has not been diagnosed with cognitive impairment, who does not exhibit any cognitive impairment symptoms, who does not have any subjective cognitive complaints (SCC) as described herein, and/or who does not have any diagnoses that puts the subject at risk of cognitive decline in the future. A cognitively normal individual who has, or would, perform at average or above average when tested using cognitive tests known to one of skill in the art. Such cognitive tests may consider, for example, factors such as age and sex when determining “average” or “above-average” performance.

In embodiments, a cognitively normal individual who remains “stable” remains a cognitively normal individual over about 1 to about 50 years, about 1 to about 30 years, about 1 to about 20 years, about 1 to about 15 years, about 1 to about 10 years, or about 1 to about 5 years. As used herein, a cognitively normal individual who “declines” refers to a cognitively normal individual as defined herein who, within about 1 to about 50 years, about 1 to about 30 years, about 1 to about 20 years, about 1 to about 15 years, about 1 to about 10 years, or about 1 to about 5 years, produces a lower test result in a cognitive test known to one of ordinary skill in the art as compared to the test result when the individual was determined as cognitively normal.

As used herein, an individual with Alzheimer's Disease (AD) refers to a person who has been diagnosed with AD using a test known to one of skill in the art, for example, clinical presentation, a biomarker test, and/or a brain scan.

As used herein, an individual with “mild cognitive impairment” or “MCI” means that the individual has a slight but noticeable decline in mental abilities, e.g., memory and thinking skills, as compared with others of the same age. The minor decline in abilities is noticeable by the individual experiencing them or by others who interact with the individual, but the changes are not severe enough to interfere with normal daily life and activities.

In embodiments, an individual with MCI who remains “stable” refers to a person who has been diagnosed with MCI using a test known to one of skill in the art and who maintains this diagnoses, e.g., over about 1 to about 50 years, about 1 to about 30 years, about 1 to about 20 years, about 1 to about 15 years, about 1 to about 10 years, or about 1 to about 5 years. As used herein, an individual with MCI who “declines” refers to a person who has been diagnosed with MCI using a cognitive test known to one of skill in the art who, within about 1 to about 50 years, about 1 to about 30 years, about 1 to about 20 years, about 1 to about 15 years, about 1 to about 10 years, or about 1 to about 5 years, produces a lower test result when tested using the cognitive test.

As used herein, “severe cognitive impairment” (used interchangeably herein with “dementia”) refers to a decline in mental function that is severe enough to interfere with daily living.

As used herein, “subjective cognitive complaints” (SCC) refer to memory and related cognitive concerns expressed by individuals with or without objective evidence of cognitive impairment. An individual with SCC may be diagnosed with MCI, severe cognitive impairment, and/or AD. See, e.g., Mitchell, Age Ageing 37(5):497-499 (2008). Methods of diagnosing individuals as cognitively normal or cognitively impaired are further described herein.

Reference throughout the present disclosure to “tau,” without specifying its phosphorylation state, encompasses phosphorylated and non-phosphorylated tau.

### Tau

In embodiments, the present disclosure provides methods for detecting and/or quantifying the amount of phosphorylated tau (referred to herein as “pTau” or “p-tau”) in a sample, wherein the pTau is phosphorylated at amino acid position T181 (pTau181), or wherein the pTau is phosphorylated at amino acid position T217 (pTau217). In embodiments, the present disclosure further provides methods for detecting and/or quantifying the amount of total tau in a sample, wherein the total tau comprises non-phosphorylated tau and pTau, and wherein the pTau is phosphorylated at any of its serine (Ser) or threonine (Thr) residues. In embodiments, the pTau is phosphorylated at amino acid position T175, T181, T212, S214, T217, cis T231, trans T231, S293, S396, S610, or a combination thereof, wherein the amino acid position corresponds to SEQ ID NO:1. Unless otherwise specified, all amino acid residues of tau or p-tau referenced herein correspond to SEQ ID NO:1. In embodiments, the pTau is phosphorylated at amino acid position T181 (referred to herein as “pTau181”). In embodiments, the pTau is phosphorylated at amino acid position T217 (referred to herein as “pTau217”).

The tau protein can be found in neurons of the central nervous system (CNS) and, to a lesser extent, in CNS astrocytes and oligodendrocytes. Tau is primarily involved in maintaining the stability of microtubules in axons by interacting with tubulin and promoting tubulin assembly into microtubules. Tau has also been shown to play a role in cellular signaling protein recruitment, microtubule-mediated axonal transport regulation, neuronal development, neuroprotection, and apoptosis. Wang et al., Prog Neurobiol 85(2):148-175 (2008); Papanikolopoulou et al., J Neurosci 39(42):8315-8329 (2019).

Tau is a phosphoprotein with 79 potential Ser and Thr phosphorylation sites on the longest tau isoform; phosphorylation has been observed at about 30 of these sites. Phosphorylation of tau is regulated by a host of kinases, including protein kinase N1 (PKN). When PKN is activated, it phosphorylates tau, resulting in disruption of microtubule organization. Taniguchi et al., J Biol Chem 276 (13):10025-10031 (2001). Cellular phosphatases also play a role in regulating the phosphorylation of tau. For example, protein phosphatases 2A (PP2A) and 2B (PP2B) are both present in human brain tissue and have the ability to dephosphorylate Ser396 of tau. The binding of these phosphatases to tau can affect tau's association with microtubules. Matsuo et al., Neuron 13(4):989-1002 (1994). Accumulation of hyperphosphorylated tau in neurons leads to neurofibrillary degeneration, which can cause various toxic effects. Hyperphosphorylation of tau can result in the self-assembly of tangles of paired helical filaments and straight filaments, which are involved in the pathogenesis of Alzheimer's disease, frontotemporal dementia, and other tauopathies. Alonso et al., Proc Natl Acad Sci USA 98(12):6923-6928 (2001); McKee et al., Brain 136(Pt 1):43-64 (2013); Omalu et al., Neurosurgery 57(1):128-134 (2005); Stern et al., N Engl J Med 380(18):1716-1725 (2019). Measurement of tau is further discussed in, e.g., Palmqvist et al., Nat Med. 27(6):1034-1042 (2021); Tissot et al., Alzheimers Res Ther. 13(1):69 (2021); Clark et al., Alzheimers Res Ther. 13(1):65 (2021); Ding et al., Transl Neurodegener. 10(1):10 (2021); Moscoso et al., Brain. 144(1):325-339 (2021); Karikari et al., Mol Psychiatry. 26(2):429-442 (2021); Lantero et al., Acta Neuropathol. 140(3):267-278 (2020); Karikari et al., Lancet Neurol. 19(5):422-433 (2020); Thijssen et al., Nat Med. 26(3):387-397 (2020); Janelidze et al., Nat Med. 26(3):379-386 (2020).

As discussed herein, diagnosis of early stage Alzheimer's disease (AD) is valuable for early and more effective treatment and for enrolling patients into clinical trials. Early stage diagnosis is also important for identifying individuals for whom treatment would likely provide the greatest benefit. In embodiments, the present disclosure provides a method of measuring the levels of pTau that is phosphorylated at a single amino acid position, T181, or T217. In embodiments, the present disclosure provides methods of measuring the levels of total tau, i.e., non-phosphorylated tau and pTau, wherein the pTau is phosphorylated at any of its serine (Ser) or threonine (Thr) residues, including T181 and/or T217.

It was discovered that the methods described herein provide a highly accurate method of determining cognitive impairment progression in an individual by utilizing a simple, convenient, sensitive, and specific method with a wide dynamic range for detecting the amount of tau, e.g., pTau181, pTau217, or total tau. Unexpectedly, the methods provided herein are capable of accurately distinguishing between (i) cognitively normal individuals who do not progress to any cognitive impairment during their lifetime (i.e., individuals whose diagnoses or symptoms remain “stable”), and (ii) cognitively normal individuals who later progress to mild cognitive impairment (MCI) during their lifetime (i.e., individuals whose diagnoses or symptoms “decline”). Advantageously, the methods provided herein are capable of determining an individual as being likely to experience cognitive decline even before the cognitive decline is detected by neurocognitive testing and/or PET imaging. Further, the methods provided herein are capable of accurately distinguishing between (i) individuals with mild MCI who do not progress to any further cognitive impairment during their lifetime (stable), and (ii) individuals with mild MCI who later progress to dementia during their lifetime (decline). In embodiments, the methods provided herein are capable of accurately distinguishing between (i) individuals with SCC who do not progress to any further cognitive impairment during their lifetime (stable), and (ii) individuals with SCC who later progress to dementia during their lifetime (decline). In embodiments, the methods provided herein are capable of accurately distinguishing between (i) cognitively normal individuals and (ii) individuals who have AD. In embodiments, the methods provided herein are capable of accurately distinguishing between (i) individuals who have AD and (ii) individuals who have a non-AD neurodegenerative disease. In embodiments, the non-AD neurodegenerative disease is frontotemporal dementia, progressive supranuclear palsy, Lewy body dementia, Pick's disease, cerebrovascular disease, amyotrophic lateral sclerosis, corticobasal degeneration, Creutzfeldt-Jakob disease, cerebral amyloid angiopathy, multiple sclerosis, thalamic degeneration, or dementia lacking distinctive histology. In contrast with prior studies referenced herein that only assess a patient population's likelihood of cognitive impairment, the methods herein are capable of providing both a cognitive impairment likelihood assessment of an individual and of a patient population. In embodiments, the methods herein monitor or track an individual subject's tau levels, e.g., pTau181, pTau217, or total tau, e.g., once every 1 month, 3 months, 6 months, 9 months, 1 year, 2 years, 3 years, 5 years, or more, over the course of 1 year, 2 years, 3 years, 4 years, 5 years, 6 years, 7 years, 8 years, 9 years, 10 years, or more than 10 years, or over the course of the subject's lifetime. For example, an individual subject can be tested every 1, 2, 3, 4, 5, 6, 7, 8, 9, 10, or more years using the methods described herein to screen for an increased risk of developing MCI and/or AD and/or increased risk of cognitive decline. In embodiments, the methods track the individual's tau levels, e.g., pTau181, pTau217, or total tau, thereby allowing for early detection, intervention, and treatment of AD.

The present methods are advantageously capable of distinguishing between patient population groups with high accuracy. As used herein, a “normalized” concentration of a particular biomarker, e.g., pTau181, pTau217, or total tau, is the average concentration of the biomarker as measured in a population. In embodiments, when comparing biomarker levels of a subject to a normalized concentration, the normalized concentration is determined in a cohort to which the subject belongs. In embodiments, the cohort is determined based on the subject's gender, age group, preexisting health condition(s), family history, genetic factors, levels of other biomarkers, or combinations thereof. An exemplary genetic factor is whether the subject has a genetic variant in the apolipoprotein E (APOE) gene, also known as the subject's APOE status. APOE status is further described, e.g., in Lumsden et al., eBioMedicine 59:102954 (2020). Further exemplary genetic factors include, but are not limited to, mutations in the subject's amyloid precursor protein (APP), presenilin 1 (PSEN1), and presenilin 2 (PSEN2). In embodiments, the normalized pTau181 and/or pTau217 concentration is normalized for age and sex of the cohort. In embodiments, the normalized pTau181 and/or pTau217 concentration is normalized for age, sex, and APOE status of the cohort.

In embodiments, the methods herein provide a receiver-operating characteristic (ROC) curve with an area-under-the-curve (AUC) value of greater than about 0.7, greater than about 0.75, greater than about 0.8, greater than about 0.85, greater than about 0.9, or greater than about 0.95 for distinguishing between the following groups: (i) cognitively normal individuals and (ii) individuals who have AD; (i) cognitively normal individuals who remain stable and (ii) cognitively normal individuals who decline; (i) individuals with SCC who remain stable and (ii) individuals with SCC who decline; and (i) individuals with mild MCI who remain stable and (ii) individuals with mild MCI who decline. In general, ROC curves and their AUC values provide an assessment of the diagnostic accuracy of a given test. In embodiments, the ROC curve is generated from a logistic regression model that includes the individuals' age and sex. In embodiments, the ROC curve is determined from a logistic regression model that includes the individuals' age, sex, and APOE status. In embodiments, the methods provided herein are capable of determining, with a positive likelihood ratio of about 5, that an individual likely has AD based on a pTau181 and/or pTau217 concentration of higher than or about 1.2-fold relative to a normalized pTau181 and/or pTau217 concentration. In embodiments, the methods provided herein are capable of determining, with a positive likelihood ratio of about 10, that an individual has AD based on a pTau181 and/or pTau217 concentration of higher than or about 2-fold relative to a normalized pTau181 and/or pTau217 concentration. In embodiments, the methods provided herein are capable of determining, with a positive likelihood ratio of about 5, that an individual with MCI is likely to decline based on a pTau181 and/or pTau217 concentration of higher than or about 2-fold relative to a normalized pTau181 and/or pTau217 concentration. In embodiments, the methods provided herein are capable of determining, with a negative likelihood ratio of about 0.1, that an individual likely does not have AD based on a pTau181 and/or pTau217 concentration of lower than or about 0.5-fold relative to a normalized pTau181 and/or pTau217 concentration. In embodiments, the methods provided herein are capable of determining, with a negative likelihood ratio of about 0.1, that an individual with MCI is likely to remain stable based on a pTau181 and/or pTau217 concentration of less than or about 0.75-fold relative to a normalized pTau181 and/or pTau217 concentration. In embodiments, the methods provided herein are capable of determining, with a positive likelihood ratio of at least about 5 or at least about 10, that an individual with SCC is likely to decline based on a pTau181 and/or pTau217 concentration of higher than or about 1.5-fold, higher than or about 1.6-fold, higher than or about 1.7-fold, higher than or about 1.8-fold, higher than or about 1.9-fold, or higher than or about 2-fold relative to a normalized pTau181 and/or pTau217 concentration. In embodiments, the methods provided herein are capable of determining, with a negative likelihood ratio of less than about 0.2 or less than about 0.1, that an individual with SCC is likely to remain stable based on a pTau181 and/or pTau217 concentration of less than or about 0.9-fold, less than or about 0.85-fold, less than or about 0.8-fold, less than or about 0.75-fold, less than or about 0.7-fold, less than or about 0.65-fold, less than or about 0.6-fold, less than or about 0.55-fold, or less than or about 0.5-fold relative to a normalized pTau181 and/or pTau217 concentration.

As used throughout this disclosure in reference to diagnostic testing, a positive likelihood ratio refers to the change in the likelihood of having a diagnosis in a subject with a positive test. For example, a positive likelihood ratio of 10 indicates a 10-fold increase in the odds of having a particular condition, e.g., AD, in a subject with a positive result. As used herein, a negative likelihood ratio refers to the change in the likelihood of having a diagnosis in a subject with a negative test. For example, a negative likelihood ratio of 0.1 indicates a 10-fold decrease in the odds of having a particular condition, e.g., AD, in a subject with a negative result. In general, tests with a positive likelihood ratio of about 5 to about 10 and/or a negative likelihood ratio of about 0.1 to about 0.2 are considered clinically useful.

In embodiments, the disclosure provides a method of detecting phosphorylated tau (pTau) in a sample, wherein the pTau is phosphorylated at amino acid position T181 (pTau181), or amino acid position T217 (pTau217), comprising: (a) contacting the sample with: (i) a first capture reagent that binds tau; (ii) a second capture reagent that binds tau; and (iii) a detection reagent that binds pTau181 or pTau217, thereby forming a complex comprising the first and second capture reagents, pTau181 or pTau217, and the detection reagent; and (b) detecting the complex, thereby detecting the pTau181 or pTau217. In embodiments, the sample is from a subject diagnosed with mild cognitive impairment (MCI) or at risk of developing MCI. In embodiments, the sample is from a subject with subjective cognitive complaints (SCC). In embodiments, the level of pTau181 in the sample is about 0.1% to about 95%, or about 0.1% to about 90%, or about 1% to about 85%, or about 5% to about 80%, or about 10% to about 75%, or about 12% to about 72%, or about 15% to about 70%, or about 18% to about 68%, or about 20% to about 65%, or about 25% to about 60% of a normalized pTau181 and/or pTau217 concentration. In embodiments, the sample comprises greater than 120% pTau181 and/or pTau217 as compared to a normalized pTau181 and/or pTau217 concentration. In embodiments, the level of pTau181 and/or pTau217 in the sample is about 105% to about 10000%, or about 110% to about 10000%, or about 110% to about 5000%, or about 120% to about 5000%, or about 115% to about 4000%, or about 120% to about 3000%, or about 125% to about 2500%, or about 150% to about 2000%, or about 175% to about 1500%, or about 200% to about 1000% of a normalized pTau181 and/or pTau217 concentration. In embodiments, the sample comprises less than 75% pTau181 and/or pTau217 as compared to a normalized pTau181 and/or pTau217 concentration. In embodiments, the sample comprises greater than 120% pTau181 and/or pTau217 as compared to a normalized pTau181 and/or pTau217 concentration.

In embodiments, the disclosure provides a method of detecting pTau181 in a sample, comprising: (a) contacting the sample with: (i) a first capture reagent that binds tau; (ii) a second capture reagent that binds tau; (iii) a first detection reagent that binds tau; and (iv) a second detection reagent that binds pTau181, thereby forming a complex comprising the first and second capture reagents, pTau181, and the first and second detection reagents; and (b) detecting the complex, thereby detecting pTau181. In embodiments, the sample is from a subject diagnosed with mild cognitive impairment (MCI) or at risk of developing MCI. In embodiments, the sample is from a subject with subjective cognitive complaints (SCC). In embodiments, the level of pTau181 in the sample is about 0.1% to about 95%, or about 0.1% to about 90%, or about 1% to about 85%, or about 5% to about 80%, or about 10% to about 75%, or about 12% to about 72%, or about 15% to about 70%, or about 18% to about 68%, or about 20% to about 65%, or about 25% to about 60% of a normalized pTau181 concentration. In embodiments, the sample comprises greater than 120% pTau181 as compared to a normalized pTau181 concentration. In embodiments, the level of pTau181 in the sample is about 105% to about 10000%, or about 110% to about 10000%, or about 110% to about 5000%, or about 120% to about 5000%, or about 115% to about 4000%, or about 120% to about 3000%, or about 125% to about 2500%, or about 150% to about 2000%, or about 175% to about 1500%, or about 200% to about 1000% of a normalized pTau181 concentration. In embodiments, the sample comprises less than 75% pTau181 as compared to a normalized pTau181 concentration. In embodiments, the sample comprises greater than 120% pTau181 as compared to a normalized pTau181 concentration.

In embodiments, the level of pTau181 and/or pTau217 is measured by performing an electrochemiluminescence (ECL) assay on a plate reading system, e.g., a MESO SCALE DIAGNOSTICS® plate reading system such as the MESO SECTOR S 600® or MESO QUICKPLEX SQ 120®. In embodiments, the level of pTau181 and/or pTau217 is measured using a MESO SCALE DIAGNOSTICS® ECL assay platform. In embodiments, the level of pTau181 is measured using MESO SCALE DIAGNOSTICS® Kit Catalog No. K151AGMS.

In embodiments, the disclosure provides a method of detecting total tau in a sample, comprising: (a) contacting the sample with: (i) a first capture reagent that binds tau; (ii) a second capture reagent that binds tau; and (iii) a detection reagent that binds tau, thereby forming a complex comprising the first and second capture reagents, tau, and the detection reagent; and b) detecting the complex, thereby detecting total tau. In embodiments, the sample is from a subject diagnosed with mild cognitive impairment (MCI) or at risk of developing MCI. In embodiments, the sample is from a subject with subjective cognitive complaints (SCC).

In embodiments, the disclosure provides a method of detecting total tau in a sample, comprising: (a) contacting the sample with: (i) a first capture reagent that binds tau; (ii) a second capture reagent that binds tau; (iii) a first detection reagent that binds tau; and (iv) a second detection reagent that binds tau, thereby forming a complex comprising the first and second capture reagents, tau, and the first and second detection reagents; and (b) detecting the complex, thereby detecting total tau. In embodiments, the sample is from a subject diagnosed with mild cognitive impairment (MCI) or at risk of developing MCI. In embodiments, the sample is from a subject with subjective cognitive complaints (SCC).

### Capture Reagent

In embodiments, the methods of the present disclosure utilize two capture reagents, i.e., a first capture reagent and a second capture reagent, that bind to the analyte of interest, e.g., pTau (including pTau181 and pTau217) or non-phosphorylated tau, referred to herein as a “dual-capture” assay. In embodiments, the two capture reagents of a dual-capture assay bind synergistically to the pTau or non-phosphorylated tau. As used herein, “synergistic binding” refers to a higher binding affinity when using two binding reagents compared with the sum of each binding reagent's individual binding affinity. In embodiments, the capture reagents of a dual-capture assay bind the analyte of interest, e.g., pTau (including pTau181 and pTau217) or non-phosphorylated tau, with higher affinity compared with a single capture reagent. In embodiments, the capture reagents of a dual-capture assay bind the analyte of interest, e.g., pTau (including pTau181 and pTau217) or non-phosphorylated tau, with greater specificity compared with a single capture reagent. In embodiments, a dual-capture assay has improved sensitivity and/or specificity relative to an assay utilizing a single capture reagent. In embodiments, a dual-capture assay is capable of detecting single picogram levels of pTau181, pTau217, and/or total tau in a sample. In embodiments, a dual-capture assay has a lower limit of quantitation (LLOQ) of less than 20 pg/mL, less than 18 pg/mL, less than 15 pg/mL, less than 12 pg/mL, less than 10 pg/mL, less than 5 pg/mL, or less than 1 pg/mL of pTau181, pTau217, and/or total tau.

In embodiments, the first capture reagent is an antibody, antigen, ligand, receptor, oligonucleotide, hapten, epitope, mimotope, or an aptamer. In embodiments, the first capture reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the first capture reagent comprises at least one heavy or light chain complementarity determining region (CDR) of an antibody. In embodiments, the first capture reagent comprises at least two CDRs from one or more antibodies. In embodiments, the first capture reagent is an antibody or antigen-binding fragment thereof. In embodiments, the first capture reagent binds tau. In embodiments, the first capture reagent is capable of binding to non-phosphorylated tau. In embodiments, the first capture reagent is capable of binding to pTau. In embodiments, the first capture reagent is capable of binding to both non-phosphorylated tau and pTau. In embodiments, the first capture reagent is capable of binding to pTau that is phosphorylated at T175, T181, T212, S214, T217, cis T231, trans T231, S293, S396, S610, or a combination thereof. In embodiments, the method detects pTau181, and the first capture reagent is capable of binding to pTau that is phosphorylated at T181. In embodiments, the method detects pTau217, and the first capture reagent is capable of specifically binding to pTau217.

In embodiments, the second capture reagent is an antibody, antigen, ligand, receptor, oligonucleotide, hapten, epitope, mimotope, or an aptamer. In embodiments, the second capture reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the second capture reagent comprises at least one heavy or light chain complementarity determining region (CDR) of an antibody. In embodiments, the second capture reagent comprises at least two CDRs from one or more antibodies. In embodiments, the second capture reagent is an antibody or antigen-binding fragment thereof. In embodiments, the second capture reagent binds tau. In embodiments, the second capture reagent is capable of binding to non-phosphorylated tau. In embodiments, the second capture reagent is capable of binding to pTau. In embodiments, the second capture reagent is capable of binding to both non-phosphorylated tau and pTau. In embodiments, the second capture reagent is capable of binding to pTau that is phosphorylated at T175, T181, T212, S214, T217, cis T231, trans T231, S293, S396, S610, or a combination thereof. In embodiments, the method detects pTau181, and the second capture reagent is capable of binding to pTau that is phosphorylated at T181. In embodiments, the method detects pTau217, and the second capture reagent is capable of specifically binding to tau.

In embodiments, each of the first and second capture reagents is an antibody or antigen-binding fragment thereof. In embodiments, each of the first and second capture reagent binds tau. In embodiments, the first and second capture reagents are capable of binding non-phosphorylated tau. In embodiments, the first and second capture reagents are capable of binding to pTau. In embodiments, the first and second capture reagents are capable of binding non-phosphorylated tau and pTau. In embodiments, the method detects pTau181, and the first and second capture reagents are capable of binding to pTau phosphorylated at T175, T181, T212, S214, T217, cis T231, trans T231, S293, S396, S610, or a combination thereof. In embodiments, the method detects pTau181, and the first and second capture reagents are the antibodies MSD clone VEC0983-0981 and MSD clone VEC0728-0727 (Meso Scale Discovery, Rockville, MD, USA, “MSD”). In embodiments, the method detects pTau181 and the first and second capture reagents are selected from MSD clones VEC1980-1977, VEC1980-1978, and VEC1980-1979.

In embodiments, the first and/or second capture reagents are immobilized on a surface prior to being contacted with the sample. In embodiments, one or both of the first and second capture reagents are immobilized on the surface simultaneously or substantially simultaneously as being contacted with the sample. In embodiments, the first and second capture reagents are contacted with the sample, then one or both of the first and second capture reagents are immobilized on the surface. In embodiments, the first and second capture reagents are immobilized on the surface. Immobilization of capture reagents onto the surface are further described herein.

In embodiments, the method comprises a single capture reagent. In embodiments, the method comprises a single capture reagent and detects pTau217, and the capture reagent is capable of specifically binding to pTau217. In embodiments, the method detects pTau217, and the capture reagent is antibody MSD clone VEC3006-3005. In embodiments, the method comprises detecting pTau217, wherein the capture reagent is antibody MSD clone VEC3006-3005 and the detection reagent binds tau. In embodiments, the detection reagent that binds tau is antibody MSD clone VEC1367-1366 and/or VEC2002-2001.

### Detection Reagent

In embodiments, the method comprises contacting the sample with a detection reagent or multiple detection reagents, thereby forming a complex comprising the capture reagent, or the first and second capture reagents, the tau (e.g., pTau181 or pTau217), and the detection reagent(s). In embodiments, the method comprises detecting the complex, thereby detecting pTau181, pTau217, and/or total tau. In embodiments, the method further comprises binding the complex to a surface prior to the detecting. In embodiments, the complex is bound to the surface via immobilization of the first and/or second capture reagents to the surface. In embodiments, the complex is formed on the surface. In embodiments, the complex is formed in solution, then bound to the surface. Formation of the complex and surfaces are further described herein.

In embodiments, the detection reagent is an antibody, antigen, ligand, receptor, oligonucleotide, hapten, epitope, mimotope, or an aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the detection reagent comprises at least one heavy or light chain complementarity determining region (CDR) of an antibody. In embodiments, the detection reagent comprises at least two CDRs from one or more antibodies. In embodiments, the detection reagent is an antibody or antigen-binding fragment thereof.

In embodiments where the method detects pTau181, the detection reagent specifically binds pTau181 or tau. In embodiments where the method detects pTau181, the detection reagent binds pTau181 and does not bind non-phosphorylated tau. In embodiments where the method detects pTau181, the detection reagent binds pTau181 and does not bind pTau that is not phosphorylated at amino acid position T181. Exemplary detection reagents that specifically bind pTau181 include, but are not limited to, the antibodies listed under Invitrogen catalog nos. MN1050 (see, e.g., Meredith Jr. et al., PLOS ONE 8(10): e76523 (2013)) and 701530. In embodiments, the detection reagent is the antibody MSD clone VEC1367-1366, VEC2002-2001 and/or VEC0728-0727. In embodiments, the detection reagent is the antibody MSD clone VEC1980-1978.

In embodiments, the method comprises detecting pTau217 using a single capture reagent and a single detection reagent, wherein the single detection reagent binds tau. In embodiments, the detection reagent that binds tau is MSD antibody clone VEC1367-1366 or VEC2002-2001.

In embodiments, the method comprises detecting pTau181 using a single capture reagent and a single detection reagent, wherein the single detection reagent binds tau. In embodiments, the detection reagent that binds tau is MSD antibody clone VEC1367-1366 or VEC2002-2001.

In embodiments, the method comprises detecting pTau181 using a single capture reagent and a two detection reagents, wherein at least one detection reagent binds tau. In embodiments, the first detection reagent that binds tau is MSD antibody clone VEC1367-1366 or VEC2002-2001, and the second detection reagent is MSD clone VEC0728-0727.

In embodiments where the method detects total tau, the detection reagent is capable of binding non-phosphorylated tau and/or pTau, for example, pTau that is phosphorylated at any of amino acid positions T175, T181, T212, T217, S214, cis T231, trans T231, S293, S396, S610, or a combination thereof. In embodiments, the detection reagent is the antibody MSD clone VEC1367-1366, VEC2002-2001 or VEC0728-0727.

In embodiments, the detection reagent comprises a detectable label. In embodiments, the detecting step of the method comprises measuring the amount of the detectable label. In embodiments where the complex is bound to a surface, the method comprises measuring the amount of detectable label on the surface. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels, and the detecting comprises measuring an ECL signal. Exemplary ECL labels and methods of measuring ECL signals are described, e.g., in U.S. Pat. Nos. 5,714,089; 6,136,268; 6,316,607; 6,468,741; 6,479,233; 6,808,939; and 9,499,573. In embodiments, the amount of measured detectable label, e.g., the amount of measured ECL signal, is used to determine the amount of tau, e.g., pTau181 or total tau, is present in the sample.

In embodiments, the detection reagent comprises a nucleic acid probe. In embodiments, the detecting step of the method comprises: (i) extending the nucleic acid probe to form an extended sequence; and (ii) measuring the amount of the extended sequence. In embodiments, the complex is bound to a surface prior to the detecting, and the surface further comprises an anchoring reagent. In embodiments, the detecting step of the method comprises: (i) extending the nucleic acid probe to form an extended sequence comprising an anchoring region that binds to the anchoring reagent; (ii) binding the extended sequence to the anchoring reagent; and (iii) measuring the amount of extended sequence bound to the surface. In embodiments, the amount of measured extended sequence, e.g., that is bound to the surface, is used to determine the amount of tau, e.g., pTau181 or total tau, is present in the sample.

In embodiments, the extending step comprises binding the nucleic acid probe to a template oligonucleotide and extending the nucleic acid probe by polymerase chain reaction (PCR), ligase chain reaction (LCR), strand displacement amplification (SDA), self-sustained synthetic reaction (3SR), isothermal amplification (such as, e.g., helicase-dependent amplification or rolling circle amplification (RCA)), or combination thereof. In embodiments, the nucleic acid probe is extended by PCR. In embodiments, the extending step comprises binding the nucleic acid probe of the detection reagent to a template oligonucleotide, ligating the template oligonucleotide to form a circular template oligonucleotide (e.g., by ligation of a linear template oligonucleotide to form a circular oligonucleotide), and extending the nucleic acid probe by RCA.

In embodiments, the sample is contacted with the detection reagent and the first and second capture reagents simultaneously or substantially simultaneously. In embodiments, the sample is contacted with: first, the first and second capture reagents; and second, the detection reagent. In embodiments, the sample is contacted with: first, the detection reagent, and second, the first and second capture reagents. In embodiments, the first and/or second capture reagents are immobilized on the surface prior to formation of the complex. In embodiments, the complex is formed, then the first and/or second capture reagents are immobilized to the surface.

In embodiments, the method comprises: contacting the sample with the detection reagent in solution to form a first complex; contacting the first complex with the first and second capture reagents to form a second complex that comprises the first and second capture reagents, tau (e.g., pTau, including pTau181, or non-phosphorylated tau), and the detection reagent; then, extending the nucleic acid probe as described herein. In embodiments, the method comprises: contacting the sample with the detection reagent in solution to form a first complex comprising tau (e.g., pTau, including pTau181, or non-phosphorylated tau) and the detection reagent; extending the nucleic acid probe and forming an extended sequence as described herein; then, binding the extended sequence to the surface via the anchoring reagent on the surface and/or binding the first complex to the surface via the first and/or second capture reagents.

In embodiments, the method comprises contacting the sample with a first detection reagent and a second detection reagent, thereby forming a complex comprising the first and second capture reagents, the tau (e.g., pTau181), the first detection reagent, and the second detection reagent. In embodiments, the method comprises detecting the complex, thereby detecting pTau181 and/or total tau. In embodiments, the method further comprises binding the complex to a surface prior to the detecting. In embodiments, the complex is bound to the surface via immobilization of the first and/or second capture reagents to the surface. In embodiments, the complex is formed on the surface. In embodiments, the complex is formed in solution, then bound to the surface. Formation of the complex and surfaces are further described herein.

In embodiments, the first detection reagent is an antibody, antigen, ligand, receptor, oligonucleotide, hapten, epitope, mimotope, or an aptamer. In embodiments, the first detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the first detection reagent comprises at least one heavy or light chain complementarity determining region (CDR) of an antibody. In embodiments, the first detection reagent comprises at least two CDRs from one or more antibodies. In embodiments, the first detection reagent is an antibody or antigen-binding fragment thereof. In embodiments, the first detection reagent binds tau. In embodiments, the first detection reagent is capable of binding to non-phosphorylated tau. In embodiments, the first detection reagent is capable of binding to pTau. In embodiments, the first detection reagent is capable of binding to both non-phosphorylated tau and pTau. In embodiments, the first detection reagent is capable of binding to pTau that is phosphorylated at T175, T181, T212, S214, T217, cis T231, trans T231, S293, S396, S610, or a combination thereof. In embodiments, the first detection reagent is capable of binding to pTau that is phosphorylated at T181.

In embodiments, the second detection reagent is an antibody, antigen, ligand, receptor, oligonucleotide, hapten, epitope, mimotope, or an aptamer. In embodiments, the second detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the second detection reagent comprises at least one heavy or light chain complementarity determining region (CDR) of an antibody. In embodiments, the second detection reagent comprises at least two CDRs from one or more antibodies. In embodiments, the second detection reagent is an antibody or antigen-binding fragment thereof.

In embodiments where the method detects pTau181, the second detection reagent specifically binds pTau181. In embodiments where the method detects pTau181, the second detection reagent binds pTau181 and does not bind non-phosphorylated tau. In embodiments where the method detects pTau181, the second detection reagent binds pTau181 and does not bind pTau that is not phosphorylated at amino acid position T181. In embodiments, the method utilizing a second detection reagent that binds pTau, e.g., pTau181, and that does not bind non-phosphorylated tau has improved specificity as compared to a method that utilizes a second detection reagent that is capable of binding non-phosphorylated tau.

In embodiments where the method detects total tau, the second detection reagent is capable of binding non-phosphorylated tau and/or pTau, for example, pTau that is phosphorylated at any of amino acid positions T175, T181, T212, S214, T217, cis T231, trans T231, S293, S396, S610, or a combination thereof.

In embodiments, the first detection reagent and/or the second detection reagent comprises a detectable label. In embodiments, the detecting step of the method comprises measuring the amount of the detectable label as described herein. In embodiments where the complex is bound to a surface, the method comprises measuring the amount of detectable label on the surface. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels, and the detecting comprises measuring an ECL signal. In embodiments, the amount of measured detectable label, e.g., the amount of measured ECL signal, is used to determine the amount of tau, e.g., pTau181 or total tau, is present in the sample.

In embodiments, the first detection reagent comprises a first nucleic acid probe, and the second detection reagent comprises a second nucleic acid probe. In embodiments, the detecting step of the method comprises: (i) extending the second nucleic acid probe to form an extended sequence; and (ii) measuring the amount of the extended sequence. In embodiments, the complex is bound to a surface prior to the detecting, and the surface further comprises an anchoring reagent. In embodiments, the detecting step of the method comprises: (i) extending the second nucleic acid probe to form an extended sequence comprising an anchoring region that binds to the anchoring reagent; (ii) binding the extended sequence to the anchoring reagent; and (iii) measuring the amount of extended sequence bound to the surface. In embodiments, the amount of measured extended sequence, e.g., that is bound to the surface, is used to determine the amount of tau, e.g., pTau181 or total tau, is present in the sample. Methods of extending the second nucleic acid probe are further described herein. In embodiments, the extending comprises binding the second nucleic acid probe to a template oligonucleotide and extending the second nucleic acid probe, e.g., by PCR, LCR, SDA, 3SR, an isothermal amplification method such as helicase-dependent amplification or RCA, or a combination thereof. In embodiments, the extending comprises binding the second nucleic acid probe to a template oligonucleotide, ligating the template oligonucleotide to form a circular template oligonucleotide (e.g., by ligation of a linear template oligonucleotide to form a circular oligonucleotide), and extending the second nucleic acid probe by RCA.

In embodiments, the sample is contacted with the first and second detection reagents and the first and second capture reagents simultaneously or substantially simultaneously. In embodiments, the sample is contacted with: first, the first and second capture reagents; and second, the first and second detection reagents. In embodiments, the sample is contacted with: first, the first and second detection reagents, and second, the first and second capture reagents. In embodiments, the first and/or second capture reagents are immobilized on the surface prior to formation of the complex. In embodiments, the complex is formed, then the first and/or second capture reagents are immobilized to the surface.

In embodiments, the method comprises: contacting the sample with the first and second detection reagents in solution to form a first complex; contacting the first complex with the first and second capture reagents to form a second complex that comprises the first and second capture reagents, tau (e.g., pTau, including pTau181 and pTau217, or non-phosphorylated tau), and the first and second detection reagents; then, extending the second nucleic acid probe as described herein. In embodiments, the method comprises: contacting the sample with the first and second detection reagents in solution to form a first complex comprising tau (e.g., pTau, including pTau181 and pTau217, or non-phosphorylated tau) and the first and second detection reagents; extending the nucleic acid probe and forming an extended sequence as described herein; then, binding the extended sequence to the surface via the anchoring reagent on the surface and/or binding the first complex to the surface via the first and/or second capture reagents.

In embodiments, the extended sequence, formed by extending the nucleic acid probe of the detection reagent or by extending the second nucleic acid probe of the second detection reagent as described herein, binds to the anchoring reagent on the surface. In embodiments, the anchoring reagent comprises an oligonucleotide, aptamer, aptamer ligand, antibody, antigen, ligand, receptor, hapten, epitope, or a mimotope. In embodiments, the extended sequence comprises an aptamer, and the anchoring reagent comprises a ligand for the aptamer. In embodiments, the extended sequence comprises an oligonucleotide, and the anchoring reagent comprises an oligonucleotide-binding protein that binds the oligonucleotide. In embodiments, the extended sequence comprises a hapten, and the anchoring reagent comprises an antibody specific for the hapten. In embodiments, the extended sequence comprises a receptor, and the anchoring reagent comprises a ligand for the receptor, e.g., a nucleobase conjugated to or modified with the ligand.

In embodiments, the anchoring reagent comprises an anchoring oligonucleotide. In embodiments, the anchoring oligonucleotide is a single stranded oligonucleotide. In embodiments, the extended sequence comprises an anchoring oligonucleotide complement that is complementary to the anchoring oligonucleotide. In embodiments, binding the extended sequence to the anchoring reagent comprises hybridizing the anchoring oligonucleotide complement to the anchoring oligonucleotide.

In embodiments, the anchoring oligonucleotide is a double stranded oligonucleotide. In embodiments, binding the extended sequence to the anchoring reagent comprises forming a triple helix between the anchoring oligonucleotide and the extended sequence. In embodiments, binding the extended sequence to the anchoring reagent comprises denaturing the anchoring oligonucleotide to provide a single stranded oligonucleotide region that binds the extended sequence. In embodiments, binding the extended sequence to the anchoring reagent comprises subjecting the anchoring oligonucleotide to a helicase and/or nuclease to provide an oligonucleotide region that binds the extended sequence.

In embodiments, the amount of extended sequence bound to the surface is measured. In embodiments, the amount of extended sequence is measured without binding the extended sequence to the surface, e.g., via the anchoring reagent as described herein. In embodiments, the measuring comprises contacting the extended sequence with a labeled probe that binds to the extended sequence and that comprises a detectable label. In embodiments, the labeled probe comprises a detection oligonucleotide and a detectable label, and the extended sequence comprises a detection oligonucleotide complement that is complementary to the detection oligonucleotide. In embodiments, the labeled probe comprises one or more detectable labels. Detectable labels are further described herein. In embodiments, the detectable label is capable of being measured by a measurement of light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels, and the measuring comprises measuring an ECL signal. In embodiments, the amount of measured ECL signal is used to determine the amount of tau, e.g., pTau181, pTau217, or total tau, in the sample.

In embodiments, the extended sequence comprises a labeled base, wherein the labeled base comprises a detectable label, and the measuring comprises measuring the detectable label. In embodiments, the extended sequence comprises a modified base, and the measuring comprises contacting the extended sequence with a detectable moiety that binds to the modified base and that comprises a detectable label. In embodiments, the modified base comprises an aptamer, aptamer ligand, antibody, antigen, ligand, receptor, hapten, epitope, or a mimotope, and the detectable moiety comprises a binding partner of the modified base. In embodiments, the modified base comprises avidin, streptavidin, and/or an antibody specific for biotin, and the detectable moiety comprises biotin and a detectable label. In embodiments, the modified base comprises biotin, and the detectable moiety comprises avidin, streptavidin, and/or an antibody specific for biotin and a detectable label. In embodiments, the detectable moiety comprises one or more detectable labels. Detectable labels are further described herein. In embodiments, the detectable label comprises one or more ECL labels, and the measuring comprises measuring an ECL signal. In embodiments, the amount of measured ECL signal is used to determine the amount of tau, e.g., pTau181, pTau217, or total tau, in the sample.

### Surface

In embodiments, the surface comprises a particle. In embodiments, the surface comprises a well of multi-well plate. In embodiments, the surface comprises a plurality of distinct binding domains, and the first capture reagent and the second capture reagent are located on two distinct binding domains on the surface. In embodiments, the surface comprises a plurality of distinct binding domains, and first capture reagent and the second capture reagent are located on the same binding domain on the surface. In embodiments where the surface further comprises an anchoring reagent, the first capture reagent, second capture reagent, and anchoring reagent are located on the same binding domain on the surface. In embodiments where the surface further comprises an anchoring reagent, the first capture reagent, second capture reagent, and anchoring reagent are located on two or more distinct binding domains on the surface.

In embodiments, the first and/or second capture reagents are immobilized on the surface, e.g., prior to or during the contacting step, or prior to or during the detecting step of the method as described herein. In embodiments, the first and/or second capture reagents are directly immobilized on the surface, e.g., via a covalent linkage between the capture reagent and the surface. In embodiments, the first and/or second capture reagents are indirectly immobilized on the surface via a secondary binding reagents, e.g., a targeting agent. In embodiments, the surface comprises a targeting agent, and each of the first and second capture reagents is linked to a targeting agent complement that is capable of binding to a targeting agent. In embodiments, the targeting agent complement directly binds to the targeting agent. In embodiments, the targeting agent and targeting agent complement comprise complementary oligonucleotides. In embodiments, the targeting agent and targeting agent complement comprise a binding pair selected from a receptor-ligand pair, an antigen-antibody pair, a hapten-antibody pair, an epitope-antibody pair, a mimotope-antibody pair, an aptamer-target molecule pair, hybridization partners, or an intercalator-target molecule pair. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin, streptavidin, or an antibody specific for biotin. In embodiments, the targeting agent and targeting agent complement are selected such that the targeting agent and targeting agent complement associated with the first capture reagent are substantially non-cross-reactive with the targeting agent and targeting agent complement associated with the second capture reagent. In embodiments, the targeting agent and targeting agent complement associated with the first capture reagent are cross-reactive with the targeting agent and targeting agent complement associated with the second capture reagent.

In embodiments, the detection reagent comprises a nucleic acid probe or the second detection reagent comprises a second nucleic acid probe, and the surface comprises (i) the first and second capture reagents as described herein, and (ii) an anchoring reagent. In embodiments, the anchoring reagent is directly immobilized on the surface, e.g., via a covalent linkage between the anchoring reagent and the surface. In embodiments, the anchoring reagent indirectly immobilized on the surface via a secondary binding reagents, e.g., a targeting agent as described herein. In embodiments, the targeting agent and targeting agent complement for the anchoring reagent is selected such that the targeting agent and targeting agent complement associated with the anchoring reagent are substantially non-cross-reactive with the targeting agent and targeting agent complement associated with the first and second capture reagents. In embodiments, the targeting agent and targeting complement associated with the anchoring reagent are cross-reactive with the targeting agent and targeting agent complement associated with the first and/or second capture reagents.

In embodiments, the targeting agent complement binds to the targeting agent via a targeting bridge agent, which is a binding partner of both the targeting agent and the targeting agent complement. In embodiments, the targeting bridge agent comprises multiple binding sites. In embodiments, the targeting bridge agent is streptavidin or avidin, and the targeting agent and targeting agent complement are each biotin.

### Sample

In embodiments, the sample is a biological sample. In embodiments, the sample comprises a mammalian fluid, secretion, or excretion. In embodiments, the biological sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions and excretions, or an extraction or purification therefrom, or dilution thereof. Further exemplary biological samples include but are not limited to physiological samples, samples containing suspensions of cells such as mucosal swabs, tissue aspirates, tissue homogenates, cell cultures, and cell culture supernatants. In embodiments, the biological sample is whole blood, serum, plasma, cerebrospinal fluid, urine, saliva, or an extraction or purification therefrom, or dilution thereof. In embodiments, the biological sample is serum or plasma. In embodiments, the plasma is in EDTA, heparin, or citrate. In embodiments, the biological sample is a mammalian fluid, secretion, or excretion that is known to contain a high level of tau, e.g., pTau181 and/or pTau217. In embodiments, the biological sample is a mammalian fluid, secretion, or excretion that is known to contain a low level of tau, e.g., pTau181 and/or pTau217. In embodiments, the biological sample containing the high or low levels of tau, e.g., pTau181 and/or pTau217, is a control for the methods described herein.

In embodiments, the sample is obtained from a subject, e.g., a human subject. In embodiments, the sample comprises plasma (e.g., in EDTA, heparin, citrate, or combination thereof) from a human subject. In embodiments, the sample comprises serum from a human subject.

In embodiments, the sample is obtained from a cognitively normal subject. As described herein, a “cognitively normal subject” is a subject who has not been diagnosed with cognitive impairment, who does not exhibit any cognitive impairment symptoms, who does not have any subjective cognitive complaints (SCC) as described herein, and/or who does not have any diagnoses that puts the individual at risk of cognitive decline in the future.

In embodiments, the sample is obtained from a subject who has been diagnosed with mild or severe cognitive impairment, e.g., as a result of AD. In embodiments, the sample is obtained from a subject who is at increased risk for AD, e.g., due to factors such as brain injury, family history, genetics, and the like.

As described herein, an individual with Alzheimer's Disease (AD) refers to a person who has been diagnosed with AD using a test known to one of skill in the art, for example, clinical presentation, a biomarker test, and/or a brain scan.

As described herein, an individual with “mild cognitive impairment” or “MCI” means that the individual has a slight but noticeable decline in mental abilities, e.g., memory and thinking skills, as compared with others of the same age. The minor decline in abilities is noticeable by the individual experiencing them or by others who interact with the individual, but the changes are not severe enough to interfere with normal daily life and activities.

As described herein, “severe cognitive impairment” (used interchangeably herein with “dementia”) refers to a decline in mental function that is severe enough to interfere with daily living.

As described herein, “subjective cognitive complaints” (SCC) refer to memory and related cognitive concerns expressed by individuals with or without objective evidence of cognitive impairment. An individual with SCC may be diagnosed with MCI, severe cognitive impairment, and/or AD. See, e.g., Mitchell, Age Ageing 37(5):497-499 (2008).

Methods of identifying individuals as cognitively normal or cognitively impaired, e.g., with MCI or dementia, are known to one of ordinary skill in the art, e.g., using neurological examinations, mental status testing, neuropsychological testing, computerized testing, genetic testing, brain imaging, or combination thereof. See, e.g., Neugroschl et al., Mt Sinai J Med. 78(4):596-612 (2011). For example, the CDR® Dementia Staging Instrument, developed at the Charles F. and Joanne Knight Alzheimer Disease Research Center (Knight ADRC), is a 5-point scale used to characterize six domains of cognitive and functional performance applicable to Alzheimer disease and related dementias: Memory, Orientation, Judgment & Problem Solving, Community Affairs, Home & Hobbies, and Personal Care (knightadrc.wustl.edu/cdr/cdr.htm). Other exemplary assessments for dementia include, e.g., the Mini-Mental State Exam (MMSE), the Mini-Cog test, and the Montreal Cognitive Assessment (MoCA) (mocatest.org). Classification of cognitively normal and cognitively impaired individuals is further described, e.g., in Dixon et al., J Clin Exp Neuropsychol 36(4):418-430 (2014). MCI, dementia, and AD are further described, e.g., in Peterson et al., Arch Neurol 56:303-308 (1999).

In embodiments, cognitively normal individuals have plasma pTau181 levels of about 0.7 to about 1.9 pg/mL, or about 0.75 to about 1.8 pg/mL, or about 0.8 to about 1.7 pg/mL, or about 0.85 to about 16 pg/mL. In embodiments, cognitively normal individuals have a mean plasma pTau181 level of about 1.3±0.6 pg/mL. In embodiments, cognitively normal individuals have plasma total tau levels of about 6 to about 13 pg/mL, or about 7.5 to about 12 pg/mL, or about 7 to about 11.5 pg/mL. In embodiments, cognitively normal individuals have a mean plasma total tau level of about 9±3 pg/mL. In embodiments, individuals with MCI who remain stable have plasma pTau181 levels of about 0.9 to about 2.2 pg/mL, or about 0.95 to about 2.1 pg/mL, or about 1 to about 2 pg/mL. In embodiments, individuals with MCI who remain stable have plasma total tau levels of about 6 to about 14 pg/mL, or about 7 to about 13.5 pg/mL, or about 8 to about 13 pg/mL. In embodiments, individuals with MCI who decline have plasma pTau181 levels of about 1.8 to about 4 pg/mL, or about 1.85 to about 3.9 pg/mL, or about 1.9 to about 3.8 pg/mL. In embodiments, individuals with MCI who remain stable have plasma total tau levels of about 8 to about 16 pg/mL, or about 8.5 to about 15.5 pg/mL, or about 9 to about 15 pg/mL. In embodiments, individuals with AD have plasma pTau181 levels of about 1.5 to about 3.9 pg/mL, or about 1.7 to about 3.7 pg/mL, or about 1.9 to about 3.5 pg/mL. In embodiments, individuals with AD have a mean plasma pTau181 level of about 2.7±1.2 pg/mL. In embodiments, individuals with AD have plasma total tau levels of about 8 to about 20 pg/mL, or about 8.5 to about 19 pg/mL, or about 9 to about 18 pg/mL. In embodiments, individuals with AD have a mean plasma total tau level of about 14±6 pg/mL. In embodiments, cognitively normal individuals have a mean plasma pTau217 level of less than about 10 pg/mL. In embodiments, individuals with AD have a mean plasma pTau217 level of greater than about 10 pg/mL.

In embodiments, the sample is obtained from the subject within about 10 minutes to about 50 years, about 20 minutes to about 30 years, about 30 minutes to about 10 years, about 40 minutes to about 5 years, about 50 minutes to about 1 year, about 1 hour to about 6 months, about 6 hours to about 1 month, about 12 hours to about 2 weeks, about 1 day to about 7 days, about 2 days to about 6 days, or about 3 days to about 4 days after being diagnosed with MCI, dementia, or AD. In embodiments, the biological sample is obtained from the subject about 1 minute, about 10 minutes, about 30 minutes, about 1 hour, about 3 hours, about 6 hours, about 12 hours, about 24 hours, about 2 days, about 3 days, about 4 days, about 5 days, about 6 days, about 1 week, about 2 weeks, about 1 month, about 3 months, about 6 months, about 1 year, about 2 years, about 3 years, about 5 years, about 10 years, about 20 years, about 30 years, or more than 30 years after being diagnosed with MCI, dementia, or AD. In embodiments, the sample is obtained from the subject once a week, 2 weeks, 1 month, 2 months, 3 months, 6 months, 9 months, 1 year, 2 years, 3 years, 5 years, or more, over the course of 1 year, 2 years, 3 years, 4 years, 5 years, 6 years, 7 years, 8 years, 9 years, 10 years, or more than 10 years.

Samples may be obtained from a single source described herein, or may contain a mixture from two or more sources, e.g., pooled from one or more subjects who may have been diagnosed with suspected to have, or at risk of developing MCI, dementia, or AD.

### Alzheimer's Disease, Cognitive Impairment and/or Decline

The methods herein can be used to assess with high clinical confidence the likelihood of developing Alzheimer's Disease (AD) and/or cognitive decline in cognitively normal individuals or individuals with subjective cognitive complaints (SCC) and/or the likelihood of further cognitive decline in individuals diagnosed with MCI, which allows for early intervention and/or treatment. In embodiments, the cognitive decline referred to herein is more than that predicted from the individual's age. The methods herein further accurately distinguish between individuals with AD and individuals with non-AD dementia. As discussed herein, early and accurate detection and diagnosis of AD can be highly important for proper treatment and/or selection for participation in clinical trials.

In embodiments, the disclosure provides a method of determining if a subject is likely to experience cognitive decline during the subject's lifetime. In embodiments, the disclosure provides a method of determining if a subject is likely to experience cognitive decline within 1 to 50 years, 1 to 40 years, 1 to 30 years, 2 to 25 years, 3 to 20 years, 4 to 15 years, or 5 to 10 years. In embodiments, the disclosure provides a method of determining if a subject is likely to experience cognitive decline within about 1, 2, 3, 4, 5, 6, 7, 8, 9, 10, 11, 12, 13, 14, 15, 16, 17, 18, 19, 20, or more than 20 years, or over the course of the subject's lifetime.

In embodiments, the disclosure provides a method of determining if a cognitively normal subject is likely to experience cognitive decline within about 1 to about 20 years, or about 1 to about 10 years, or about 1 to about 5 years, or about 1, 2, 3, 4, 5, 6, 7, 8, 9, or 10 years, comprising: (a) obtaining a measurement of pTau181, pTau217, and/or total tau levels of the subject; and (b) determining if the subject is likely to experience cognitive decline based on the measurement of pTau181, pTau217, and/or total tau levels. In embodiments, the disclosure provides a method of preventing, reducing, or delaying cognitive decline in a cognitively normal subject, comprising: (a) obtaining a measurement of pTau181, pTau217, and/or total tau levels of the subject; (b) identifying the subject as being likely to experience future cognitive decline, e.g., within about 1 to about 20 years, or about 1 to about 10 years, or about 1 to about 5 years, or about 1, 2, 3, 4, 5, 6, 7, 8, 9, or 10 years, based on the measurement of pTau181, pTau217, and/or total tau levels; and (c) administering a regimen to the subject to prevent, reduce, or delay cognitive decline. In embodiments, step (a) comprises obtaining a measurement of the subject's pTau181 and/or pTau217 levels. In embodiments, step (a) comprises obtaining a measurement of the subject's total tau levels. In embodiments, step (a) comprises obtaining a measurement of the levels of any combination of pTau181, pTau217, and total tau. In embodiments, the measurement of pTau181, pTau217, and/or total tau levels is measured by a method described herein. In embodiments, the method comprises determining that the cognitively normal subject is likely to experience further cognitive decline within about 1 to about 5 years, or about 1 to about 4 years, or about 1 to about 3 years, or about 1 to about 2 years, when the subject's pTau181 and/or pTau217 levels are at least 1.5-fold, at least 1.6-fold, at least 1.7-fold, at least 1.8-fold, at least 1.9-fold, at least 2-fold, at least 3-fold, at least 4-fold, or at least 5-fold higher than a normalized pTau181 and/or pTau217 concentration; and/or when the subject's total tau levels are at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, or at least 5-fold higher than a normalized total tau concentration.

In embodiments, the disclosure provides a method of determining if a subject with SCC is likely to experience cognitive decline within about 1 to about 20 years, or about 1 to about 10 years, or about 1 to about 5 years, or about 1, 2, 3, 4, 5, 6, 7, 8, 9, or 10 years, comprising: (a) obtaining a measurement of pTau181, pTau217, and/or total tau levels of the subject; and (b) determining if the subject is likely to experience cognitive decline based on the measurement of pTau181, pTau217, and/or total tau levels. In embodiments, the disclosure provides a method of preventing, reducing, or delaying cognitive decline in a subject with subjective cognitive complaints, comprising: (a) obtaining a measurement of pTau181, pTau217, and/or total tau levels of the subject; (b) identifying the subject as being likely to experience future cognitive decline, e.g., within about 1 to about 20 years, or about 1 to about 10 years, or about 1 to about 5 years, or about 1, 2, 3, 4, 5, 6, 7, 8, 9, or 10 years, based on the measurement of pTau181, pTau217, and/or total tau levels; and (c) administering a regimen to the subject to prevent, reduce, or delay cognitive decline. In embodiments, step (a) comprises obtaining a measurement of the subject's pTau181 and/or pTau217 levels. In embodiments, step (a) comprises obtaining a measurement of the subject's total tau levels. In embodiments, step (a) comprises obtaining a measurement of the levels of any combination of pTau181, pTau217, and total tau. In embodiments, the measurement of pTau181, pTau217, and/or total tau levels is measured by a method described herein. In embodiments, the method comprises determining that the subject with SCC is likely to experience cognitive decline within about 1 to about 5 years, or about 1 to about 4 years, or about 1 to about 3 years, or about 1 to about 2 years, when the subject's pTau181, pTau217, and/or total tau levels are at least 1.5-fold, at least 1.6-fold, at least 1.7-fold, at least 1.8-fold, at least 1.9-fold, at least 2-fold, at least 3-fold, at least 4-fold, or at least 5-fold higher than a normalized pTau181 and/or pTau217 concentration.

In embodiments, the disclosure provides a method of determining if a subject with MCI is likely to experience further cognitive decline within about 1 to about 20 years, or about 1 to about 10 years, or about 1 to about 5 years, or about 1, 2, 3, 4, 5, 6, 7, 8, 9, or 10 years, comprising: (a) obtaining a measurement of pTau181, pTau217, and/or total tau levels of the subject; and (b) determining if the subject is likely to experience further cognitive decline based on the measurement of pTau181, pTau217, and/or total tau levels. In embodiments, the disclosure provides a method of preventing, reducing, or delaying further cognitive decline in a subject with MCI, comprising: (a) obtaining a measurement of pTau181, pTau217, and/or total tau levels of the subject; (b) identifying the subject as being likely to experience further cognitive decline, e.g., within about 1 to about 20 years, or about 1 to about 10 years, or about 1 to about 5 years, or about 1, 2, 3, 4, 5, 6, 7, 8, 9, or 10 years, based on the measurement of pTau181, pTau217, and/or total tau levels; and (c) administering a regimen to the subject to prevent, reduce, or delay further cognitive decline. In embodiments, step (a) comprises obtaining a measurement of the subject's pTau181 and/or pTau217 levels. In embodiments, step (a) comprises obtaining a measurement of the subject's total tau levels. In embodiments, step (a) comprises obtaining a measurement of the levels of any combination of pTau181, pTau217, and total tau. In embodiments, the measurement of pTau181, pTau217, and/or total tau levels is measured by a method described herein. In embodiments, the method comprises determining that the subject with MCI is likely to experience further cognitive decline within about 1 to about 5 years, or about 1 to about 4 years, or about 1 to about 3 years, or about 1 to about 2 years, when the subject's pTau181 and/or pTau217 levels are at least 1.7-fold, at least 1.8-fold, at least 1.9-fold, at least 2-fold, at least 3-fold, at least 4-fold, or at least 5-fold higher than a normalized pTau181 and/or pTau217 concentration; and/or when the subject's total tau levels are at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, or at least 5-fold higher than a normalized total tau concentration. In embodiments, the method is capable of determining if a subject with MCI is likely to experience further cognitive decline, based on pTau181 and/or pTau217 levels of the subject, with a clinical sensitivity of at least 80%, at least 85%, at least 88%, or at least 90%, and a clinical specificity of at least 65%, at least 70%, at least 75%, at least 78%, at least 80%, at least 85%, at least 88%, or at least 90%. In embodiments, the method is capable of determining if a subject with MCI is likely to experience further cognitive decline, based on total tau levels of the subject, with a clinical sensitivity of at least 80%, at least 85%, at least 88%, or at least 90%, and a clinical specificity of at least 40%, at least 50%, at least 60%, at least 70%, at least 80%, or at least 90%.

In embodiments, the disclosure provides a method of distinguishing a subject afflicted with AD from an individual afflicted with non-Alzheimer's dementia, the method comprising: (a) obtaining a measurement of pTau181, pTau217, and/or total tau levels of the subject; and (b) identifying, based on the measurement of pTau181, pTau217, and/or total tau levels, the subject as (i) afflicted with AD or (ii) afflicted with non-Alzheimer's dementia. In embodiments, the disclosure provides a method of treating AD in a subject in need thereof, comprising: (a) obtaining a measurement of pTau181, pTau217, and/or total tau levels of the subject, wherein the measurement is obtained prior to administration of a treatment for AD; (b) determining, based on the measurement of pTau181, pTau217, and/or total tau levels, that the subject is afflicted with Alzheimer's disease; and (c) administering a treatment regimen for AD to the subject. In embodiments, step (a) comprises obtaining a measurement of the subject's pTau181 and/or pTau217 levels. In embodiments, step (a) comprises obtaining a measurement of the subject's total tau levels. In embodiments, step (a) comprises obtaining a measurement of the levels of any combination of pTau181, pTau217, and total tau. In embodiments, the measurement of pTau181, pTau217, and/or total tau levels is measured by a method described herein. In embodiments, the method comprises determining that the subject is afflicted with AD when the subject's pTau181 and/or pTau217 levels are at least 2-fold, at least 2.5-fold, at least 3-fold, at least 4-fold, or at least 5-fold higher than a normalized pTau181 and/or pTau217 concentration; and/or when the subject's total tau levels are at least 1.5-fold, at least 1.6-fold, at least 1.7-fold, at least 1.8-fold, at least 1.9-fold, or at least 2-fold higher than a normalized total tau concentration. In embodiments, the method comprises determining that the subject is afflicted with non-Alzheimer's dementia when the subject's pTau181 and/or pTau217 levels are about 1.1-fold to about 1.6-fold, or about 1.2-fold to about 1.5-fold, or about 1.3-fold to about 1.4-fold higher than a normalized pTau181 and/or pTau217 concentration; and/or when the subject's total tau levels are about 1.1-fold to about 1.4-fold, or about 1.1-fold to about 1.3-fold, or about 1.1-fold to about 1.2-fold higher than a normalized total tau concentration. In embodiments, a subject afflicted with AD has at least 1.5-fold, at least 1.6-fold, at least 1.7-fold, at least 1.8-fold, at least 1.9-fold, or at least 2-fold higher pTau181 and/or pTau217 levels and/or at least 1.2-fold, at least 1.3-fold, at least 1.4-fold, or at least 1.5-fold higher total tau levels as compared to an individual afflicted with non-Alzheimer's dementia. In embodiments, the method is capable of distinguishing a subject afflicted with AD from an individual afflicted with non-Alzheimer's dementia, based on pTau181 and/or pTau217 levels of the subject, with a clinical sensitivity of at least 75%, at least 78%, at least 80%, at least 85%, at least 88%, or at least 90%, and a clinical specificity of at least 75%, at least 78%, at least 80%, at least 85%, at least 88%, or at least 90%. In embodiments, the method is capable of distinguishing a subject afflicted with AD from an individual afflicted with non-Alzheimer's dementia, based on total tau levels of the subject, with a clinical sensitivity of at least 60%, at least 65%, at least 70%, at least 75%, at least 80%, at least 85%, or at least 90%, and a clinical specificity of at least 60%, at least 65%, at least 70%, at least 75%, at least 80%, at least 85%, or at least 90%.

In embodiments, the disclosure provides a method of monitoring response to treatment for AD in a subject, comprising: (a) obtaining a first measurement of pTau181, pTau217, and/or total tau levels of the subject, wherein the first measurement is obtained prior to administration of a treatment regimen for AD; (b) obtaining a second measurement of pTau181, pTau217, and/or total tau levels of the subject at one or more time points after administration of the treatment regimen for AD has been initiated; (c) determining, based on the first and second measurements of pTau181, pTau217, and/or total tau levels, that the subject is responding positively to the treatment regimen; and (d) continuing to administer the treatment regimen for AD to the subject. In embodiments, the one or more time points comprises about 1 hour to about 20 years, about 1 hour to about 10 years, about 12 hours to about 5 years, about 1 day to about 4years, about 3 days to about 3 years, about 5 days to about 2 years, about 7 days to about 18 months, about 10 days to about 12 months, about 2 weeks to about 11 months, about 3 weeks to about 10 months, about 1 month to about 9 months, about 2 months to about 8 months, about 3 months to about 7 months, or about 4 months to about 6 months. In embodiments, the one or more time points comprises 1, 2, 3, 4, 5, 6, 7, 8, 9, 10, or more than 10 separate time points within about 1 week, within about 1 month, within about 3 months, within about 6 months, within about 1 year, within about 5 years, or within about 10 years. Methods of assessing whether a subject is responding positively to a treatment regimen include, e.g., assessing the change in the subject's biomarker levels (e.g., pTau181, pTau217, and/or total tau) before and after the treatment using a method described herein. In embodiments, the methods provided herein for measuring biomarker levels (e.g., pTau181, pTau217, and/or total tau) provides a simpler and more cost-effective method for assessing a subject's response to treatment as compared to conventional methods, such as imaging studies or long-term evaluation of clinical symptoms.

In embodiments, the disclosure provides a method of determining eligibility of a subject to participate in a clinical trial of a therapeutic drug for preventing or delaying AD, the method comprising: (a) obtaining a measurement of pTau181, pTau217, and/or total tau levels of the subject; (b) determining the eligibility of the subject for the clinical trial based on the measurement of pTau181, pTau217, and/or total tau levels. In embodiments, the subject has been diagnosed with dementia or MCI. In embodiments, the subject has SCC. In embodiments, the subject does not have any cognitive impairment. In embodiments, the disclosure provides a method of conducting a clinical trial of a therapeutic drug or intervention for Alzheimer's disease, the method comprising: (a) obtaining a measurement of pTau181, pTau217, and/or total tau levels of a subject; (b) determining eligibility of the subject for the clinical trial based on the measurement of pTau181, pTau217, and/or total tau levels; and (c) administering the therapeutic drug to the subject. In embodiments, step (a) comprises obtaining a measurement of the subject's pTau181, pTau217, levels. In embodiments, step (a) comprises obtaining a measurement of the subject's total tau levels. In embodiments, step (a) comprises obtaining a measurement of the levels of any combination of pTau181, pTau217, and total tau. In embodiments, the measurement of pTau181, pTau217, and/or total tau levels is measured by a method described herein. A subject's eligibility in a clinical trial may be determined based on the subject's measured levels of pTau181, pTau217, and/or total tau. In embodiments, a subject is identified as eligible for a clinical trial when the subject's pTau181 and/or pTau217 is greater than 120%, greater than 150%, greater than 170%, or greater than 200% of a normalized pTau181 and/or pTau217 concentration. In embodiments, a subject is identified as ineligible for a clinical trial when the subject's pTau181 and/or pTau217 is less than 100%, less than 90%, less than 85%, less than 80%, or less than 75% of a normalized pTau181 and/or pTau217 concentration. In embodiments, an individual determined to be at low risk for cognitive impairment (e.g., when the subject's pTau181 and/or pTau217 is less than 100%, less than 90%, less than 85%, less than 80%, or less than 75% of a normalized pTau181 and/or pTau217 concentration) is included in a clinical trial as part of the clinical trial's safety evaluation (e.g., an FDA Phase I clinical trial); as a healthy control subject (e.g., an FDA Phase II and/or III clinical trial); and/or as a subject in a long-term study clinical trial (e.g., an FDA Phase IV clinical trial). Normalized concentrations are further described herein.

In embodiments, the disclosure provides a method of diagnosing AD in a subject comprising, when levels of pTau181 and/or pTau217 in the subject are higher than 120% relative to a normalized concentration of pTau181 and/or pTau217 as measured by a method described herein, diagnosing the subject with AD. In embodiments, when levels of pTau181 and/or pTau217 are less than 50% relative a normalized concentration of pTau181 and/or pTau217 as measured by a method described herein, the method comprises diagnosing the subject as not likely to develop AD. In embodiments, the disclosure provides a method of assessing risk for developing AD in a subject comprising: when levels of pTau181 and/or pTau217 in the subject are higher than 120% relative to a normalized concentration of pTau181 and/or pTau217 as measured by a method described herein, diagnosing the subject as having increased risk of developing AD; and when levels of pTau181 and/or pTau217 in the subject are lower than 50% relative to a normalized concentration of pTau181 as measured by a method described herein, diagnosing the subject as having decreased risk of developing AD. In embodiments, the disclosure provides a method of preventing, reducing, or delaying AD in a subject comprising, when levels of pTau181 and/or pTau217 in the subject are higher than 120% relative to a normalized concentration of pTau181 and/or pTau217 as measured by a method described herein, providing a regimen to the subject to prevent, reduce, or delay AD. In embodiments, pTau181 and/or pTau217 levels of higher than 120% relative a normalized concentration of pTau181 and/or pTau217 corresponds to a positive likelihood ratio of about 5 for a method described herein. In embodiments, pTau181 and/or pTau217 levels of lower than 50% relative a normalized concentration of pTau181 and/or pTau217 corresponds to a negative likelihood ratio of about 0.1 for a method described herein. Positive and negative likelihood ratios are further described herein.

In embodiments, the disclosure provides a method of diagnosing AD in a subject having increased likelihood of developing AD comprising, when levels of pTau181 and/or pTau217 in the subject correspond to a positive likelihood ratio of greater than 5 when determined by a method described herein, diagnosing the subject with AD. In embodiments where a subject has an increased likelihood of developing AD, e.g., due to the subject's age, gender, preexisting health conditions, family history, genetic factors (e.g., APOE status), and/or other biomarker levels, a lower-fold increase of pTau181 and/or pTau217 levels is required for providing an AD diagnosis, as compared to a subject who does not an increased likelihood of developing AD. In embodiments, the disclosure provides a method of assessing risk of developing AD in a subject having increased likelihood of developing AD comprising: when levels of pTau181 and/or pTau217 in the subject provide a positive likelihood ratio of greater than 5 when determined by a method described herein, determining the subject as having increased risk of developing AD; and when levels of pTau181 and/or pTau217 in the subject provide a negative likelihood ratio of 0.1 when determined by a method described herein, determining the subject as having decreased risk of developing AD. In embodiments, when a subject has increased likelihood of developing AD, pTau181 and/or pTau217 levels in the subject of higher about 105%, higher than about 110%, higher than about 115%, or higher than about 120% relative to a normalized concentration of pTau181 and/or pTau217 correspond to a positive likelihood ratio of about 5 for a method described herein. In embodiments, the disclosure provides a method of preventing, reducing, or delaying AD in a subject having increased likelihood of developing AD comprising, when levels of pTau181 and/or pTau217 in the subject correspond to a positive likelihood ratio of greater than 5 when determined by a method described herein, providing a regimen to the subject to prevent, reduce, or delay AD. In embodiments, the subject having increased likelihood of developing AD is 1.5×, 2×, 3×, 4×, 5×, 6×, 7×, 8×, 9×, or 10× as likely to develop AD within about 1 to about 50 years, or about 1 to about 20 years, or about 1 to about 10 years, or about 1, 2, 3, 4, 5, 6, 7, 8, 9, or 10 years as compared to a cognitively normal subject. In embodiments, the cognitively normal subject is from a same cohort as the subject having increased likelihood of developing AD. In embodiments, a subject is identified as having increased likelihood of AD due to their gender, age group, preexisting health condition(s), family history, genetic factors (e.g., APOE status), prior diagnostic testing (e.g., levels of other biomarkers), or combinations thereof.

In embodiments, the disclosure provides a method of predicting cognitive decline in a subject with MCI comprising: when levels of pTau181 and/or pTau217 in the subject are higher than 200% relative a normalized concentration of pTau181 and/or pTau217 as measured by a method described herein, diagnosing the subject as having increased risk of cognitive decline; and when levels of pTau181 and/or pTau217 in the subject are lower than 75% relative to a normalized concentration of pTau181 and/or pTau217 as measured by a method described herein, diagnosing the subject as having decreased risk of cognitive decline. In embodiments, the disclosure provides a method of preventing, reducing, or delaying cognitive decline in a subject with MCI comprising, when levels of pTau181 in the subject are higher than 200% relative to a normalized concentration of pTau181 and/or pTau217 as measured by a method described herein, providing a regimen to the subject to prevent, reduce, or delay cognitive decline. In embodiments, pTau181 and/or pTau217 levels of higher than 200% relative a normalized concentration of pTau181 and/or pTau217 corresponds to a positive likelihood ratio of about 5 for a method described herein. In embodiments, pTau181 and/or pTau217 levels of lower than 75% relative to a normalized concentration of pTau181 and/or pTau217 corresponds to a negative likelihood ratio of about 0.1 for a method described herein. MCI, cognitive decline, and positive and negative likelihood ratios are further described herein.

In embodiments, the disclosure provides a method of predicting cognitive decline in a subject with MCI comprising: when levels of pTau181 and/or pTau217 in the subject provide a positive likelihood ratio of greater than 5 when determined by a method described herein, determining the subject as having increased risk of cognitive decline; and when levels of pTau181 and/or pTau217 in the subject provide a negative likelihood ratio of about 0.1 to about 0.2 when determined by a method described herein, determining the subject as having decreased risk of cognitive decline. In embodiments, the disclosure provides a method of preventing, reducing, or delaying cognitive decline in a subject with MCI comprising, when levels of pTau181 and/or pTau217 in the subject correspond to a positive likelihood ratio of greater than 5 when determined by a method described herein, providing a regimen to the subject to prevent, reduce, or delay cognitive decline. In embodiments, pTau181 and/or pTau217 levels of higher than 200% relative a normalized concentration of pTau181 and/or pTau217 corresponds to a positive likelihood ratio of about 5 for a method described herein. In embodiments, pTau181 and/or pTau217 levels of lower than 75% relative to a normalized concentration of pTau181 and/or pTau217 corresponds to a negative likelihood ratio of about 0.1 for a method described herein.

In embodiments, the method for detecting pTau181 comprises (a) contacting a sample from the subject with: (i) a first capture reagent that binds tau; (ii) a second capture reagent that binds tau, wherein the first and second capture reagents are on a surface; and (iii) one or more detection reagents that binds pTau181, thereby forming a complex on the surface comprising the first and second capture reagents, pTau181, and the one or more detection reagents; and (b) detecting the complex on the surface, as described herein.

In embodiments, the method for detecting pTau217 comprises (a) contacting a sample from the subject with: (i) a capture reagent that binds pTau217; wherein the capture reagent is on a surface; and (iii) one or more detection reagents that is capable of binding tau, thereby forming a complex on the surface comprising the capture reagent, pTau217, and the one or more detection reagents; and (b) detecting the complex on the surface, as described herein.

In embodiments, the method for detecting total tau comprises (a) contacting a sample from the subject with: (i) a first capture reagent that binds tau; (ii) a second capture reagent that binds tau, wherein the first and second capture reagents are on a surface; and (iii) one or more detection reagents that binds tau, thereby forming a complex on the surface comprising the first and second capture reagents, tau, and the one or more detection reagents; and (b) detecting the complex on the surface, as described herein.

Treatment regimens for AD are known to one of ordinary skill in the art. In embodiments, the treatment regimen for AD comprises one or more drugs that slow disease progression and/or mitigate one or more symptoms of AD, including cognitive and non-cognitive symptoms. In embodiments, the treatment regime comprises administering an immunotherapy drug, such as a monoclonal antibody. In embodiments, the monoclonal antibody is IgG1. In embodiments, the monoclonal antibody is a humanized antibody. In embodiments, the immunotherapy drug is lecanemab (LEQEMBI®), gantenerumab, or donanemab, An exemplary drug that slows AD progression is aducanumab, also known by its tradename ADUHELM™. Non-limiting examples of drugs for mitigation of AD cognitive symptoms include cholinesterase inhibitors, e.g., donepezil (ARICEPT®), rivastigmine (EXELON®), and galantamine (RAZADYNE®); glutamate regulators, e.g., memantine (NAMENDA®); or combination therapies such as NAMZARIC®, a combination of donepezil and memantine. Non-limiting examples of drugs for mitigation of AD behavioral and psychological symptoms include orexin receptor antagonists, e.g., suvorexant (BELSOMRAR). Further drugs that may be comprised in the treatment regimen for AD include, for example, sleep aids, anti-anxiety drugs, anti-convulsants, and/or antipsychotics.

In embodiments, the treatment regimen for AD comprises a non-drug therapeutic, e.g., light therapy using a photobiomodulation (PBM) device as described in Dougal et al., Photomodulation, Photomedicine, and Laser Surgery 39(10):654-660 (2021). In embodiments, the treatment regimen for AD comprises treatments of sleep disorders, e.g., bright light and melatonin therapy, anti-depressant hypnotic therapy, continuous positive airway pressure, acoustic stimulation, and transcranial alternating current stimulation as described in Kent et al., Progress in Neurobiology 197:101902 (2021). In embodiments, the treatment regimen for AD comprises active and passive immunotherapy treatments, e.g., anti-Aβ immunotherapies as described in Spencer and Masliah, Frontiers in Aging Neuroscience, 6:114 (2014). In embodiments, the treatment regimen for AD comprises stem cell therapies using embryonic stem cells, induced pluripotent stem cells, mesenchymal stem cells treatments, or combinations thereof as described in Athar et al., Molecular Biology Reports 48(7):5629-5645 (2021). In embodiments, the treatment regimen for AD comprises therapeutics targeting secretases including β-secretase inhibitors, γ-secretase inhibitors, α-secretase stimulators as described in Athar et al., (2021). In embodiments, the treatment regimen for AD comprises treatments targeting exosomes as described in Zhang et al., Ageing Research Reviews 68:101321 (2021).

### Kits

In embodiments, the disclosure provides a kit for detecting pTau181 comprising, in one or more vials, containers, or compartments: (a) a first capture reagent that binds tau; (b) a second capture reagent that binds tau; (b) a detection reagent that binds pTau181; and (d) optionally a surface, wherein the first and second capture reagents are provided on the surface or are capable of binding to the surface. In embodiments, the kit comprises the surface. In embodiments, the kit does not comprise a surface.

In embodiments, the disclosure provides a kit for detecting pTau181 comprising, in one or more vials, containers, or compartments: (a) a first capture reagent that binds tau; (b) a second capture reagent that binds tau; (c) a first detection reagent that binds tau; (d) a second detection reagent that binds pTau181; and (e) optionally a surface, wherein the first and second capture reagents are provided on the surface or are capable of binding to the surface. In embodiments, the kit comprises the surface. In embodiments, the kit does not comprise a surface.

In embodiments, the disclosure provides a kit for detecting pTau217 comprising, in one or more vials, containers, or compartments: (a) a capture reagent that binds pTau217; (b) a detection reagent that binds tau; and (d) optionally a surface, wherein the capture reagent is provided on the surface or are capable of binding to the surface. In embodiments, the kit comprises the surface. In embodiments, the kit does not comprise a surface. In embodiments, the kit comprises a capture reagent that is MSD antibody clone VEC3006-3005 and a detection reagent that is MSD antibody clone VEC1367-1366 and/or VEC2002-2001.

In embodiments, the disclosure provides a kit for detecting total tau comprising, in one or more vials, containers, or compartments: (a) a first capture reagent that binds tau; (b) a second capture reagent that binds tau; (c) a detection reagent that binds tau; and (d) optionally a surface, wherein the first and second capture reagents are provided on the surface or are capable of binding to the surface. In embodiments, the first and second capture reagents and the detection reagent are capable of binding non-phosphorylated tau and/or pTau. In embodiments, the kit comprises the surface. In embodiments, the kit does not comprise a surface.

In embodiments, the disclosure provides a kit for detecting total tau comprising, in one or more vials, containers, or compartments: (a) a first capture reagent that binds tau; (b) a second capture reagent that binds tau; (c) a first detection reagent that binds tau; (d) a second detection reagent that binds tau; and (e) optionally a surface, wherein the first and second capture reagents are provided on the surface or are capable of binding to the surface. In embodiments, the first and second capture reagents and the first and second detection reagents are capable of binding non-phosphorylated tau and/or pTau. In embodiments, the kit comprises the surface. In embodiments, the kit does not comprise a surface.

Capture reagents are described herein. In embodiments, the first capture reagent and the second capture reagent are each independently an antibody, antigen, ligand, receptor, oligonucleotide, hapten, epitope, mimotope, or an aptamer. In embodiments, the first capture reagent and the second capture reagent are each independently an antibody or a variant thereof, including an antigen/epitope-binding portion thereof, an antibody fragment or derivative, an antibody analogue, an engineered antibody, or a substance that binds to antigens in a similar manner to antibodies. In embodiments, the first capture reagent and the second capture reagent each independently comprises at least one heavy or light chain complementarity determining region (CDR) of an antibody. In embodiments, the first capture reagent and the second capture reagent each independently comprises at least two CDRs from one or more antibodies. In embodiments, the first capture reagent and the second capture reagent are each independently an antibody or antigen-binding fragment thereof.

In embodiments, the first capture reagent and/or the second capture reagent binds tau. In embodiments, the first capture reagent and/or the second capture reagent is capable of binding to non-phosphorylated tau. In embodiments, the first capture reagent and/or the second capture reagent is capable of binding to pTau. In embodiments, the first capture reagent and/or the second detection reagent is capable of binding to both non-phosphorylated tau and pTau. In embodiments, the first capture reagent and/or the second capture reagent is capable of binding to pTau that is phosphorylated at T175, T181, T212, S214, T217, cis T231, trans T231, S293, S396, S610, or a combination thereof. In embodiments, the first capture reagent and/or the second capture reagent is capable of binding to pTau that is phosphorylated at T181.

In embodiments, each of the first and second capture reagents is an antibody or antigen-binding fragment thereof. In embodiments, each of the first and second capture reagents binds tau. In embodiments, the first and second capture reagents are capable of binding non-phosphorylated tau. In embodiments, the first and second capture reagents are capable of binding to pTau. In embodiments, the first and second capture reagents are capable of binding non-phosphorylated tau and pTau. In embodiments, the kit detects pTau181, and the first and second capture reagents are capable of binding to pTau phosphorylated at T175, T181, T212, S214, T217, cis T231, trans T231, S293, S396, S610, or a combination thereof. In embodiments, the kit detects pTau181, and the first and second capture reagents are the antibodies MSD clone VEC0983-0981 and MSD clone VEC0728-0727.

In embodiments, the kit comprises a surface, and the first capture reagent and/or the second capture reagent are immobilized on the surface. In embodiments, the kit comprises a surface, and the first capture reagent and/or the second capture reagent are capable of being immobilized to the surface. In embodiments, the kit further comprises a reagent for immobilizing the first and/or second capture reagents to the surface. Immobilization of capture reagents onto surfaces is described herein. In embodiments, the first capture reagent and/or the second capture reagent are directly immobilized onto the surface. In embodiments, the first capture reagent and/or the second capture reagent are indirectly immobilized onto the surface, e.g., via secondary binding reagents. Secondary binding reagents, e.g., targeting agents, targeting agent complements, and bridging agents, are further described herein.

In embodiments, the kit comprises a detection reagent. Detection reagents are described herein. In embodiments, the detection reagent is an antibody, antigen, ligand, receptor, oligonucleotide, hapten, epitope, mimotope, or an aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the detection reagent comprises at least one heavy or light chain complementarity determining region (CDR) of an antibody. In embodiments, the detection reagent comprises at least two CDRs from one or more antibodies. In embodiments, the detection reagent is an antibody or antigen-binding fragment thereof.

In embodiments where the kit is for detecting pTau181, the detection reagent specifically binds pTau181. In embodiments where the method detects pTau181, the detection reagent binds pTau181 and does not bind non-phosphorylated tau. In embodiments where the method detects pTau181, the detection reagent binds pTau181 and does not bind pTau that is not phosphorylated at amino acid position T181. Exemplary detection reagents that specifically bind pTau181 include, but are not limited to, the antibodies listed under Invitrogen catalog nos. MN1050 (see, e.g., Meredith Jr. et al., PLOS ONE 8(10): e76523 (2013)) and 701530. In embodiments, the detection reagent is the antibody MSD clone VEC1367-1366, VEC2002-2001 and/or VEC0728-0727. In embodiments, the detection reagent is the antibody MSD clone VEC1980-1978.

In embodiments where the kit is for detecting total tau, the detection reagent is capable of binding non-phosphorylated tau and/or pTau, for example, pTau that is phosphorylated at any of amino acid positions T175, T181, T212, S214, T217, cis T231, trans T231, S293, S396, S610, or a combination thereof. In embodiments, the detection reagent is the antibody MSD clone VEC1367-1366, VEC2002-2001 and/or VEC0728-0727.

In embodiments, the detection reagent comprises a detectable label. In embodiments, the detection reagent is capable of being conjugated to a detectable label, and the kit further comprises the detectable label and/or a reagent for performing the conjugation. Methods of conjugating detectable labels to detection reagents are known to one of ordinary skill in the art. In embodiments, the detection reagent comprises a first reactive group, and the detectable label comprises a second reactive group that is capable of reacting with the first reactive group. Non-limiting examples of first and second reactive groups include: amine and N-hydroxysuccinimide (NHS) ester; thiol and maleimide; thiol and iodoacetamide; thiol and activated disulfide; alkene or strained alkene and tetrazine; and alkyne or strained alkyne and azide.

Detectable labels are further described herein. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels.

In embodiments, the detection reagent comprises a nucleic acid probe. In embodiments, the detection reagent is capable of being conjugated to a nucleic acid probe, and the kit further comprises the nucleic acid probe and/or a reagent for performing the conjugation. Methods of conjugating nucleic acid probes to detection reagents are known to one of ordinary skill in the art and are described, e.g., in WO 2020/180645. In embodiments, the detection reagent comprises a first reactive group, and the nucleic acid probe comprises a second reactive group that is capable of reacting with the first reactive group. In embodiments, the reagent for performing the conjugation comprises a first reactive group that is capable of reacting with the detection reagent and a second reactive group that is capable of reacting with the nucleic acid probe. Examples of reactive groups and their reaction partners include but are not limited to: amine and N-hydroxysuccinimide (NHS) ester; thiol and maleimide; thiol and iodoacetamide; thiol and activated disulfide; alkene or strained alkene and tetrazine; and alkyne or strained alkyne and azide.

In embodiments, the detection reagent comprises a nucleic acid probe, and the kit further comprises an anchoring reagent. Anchoring reagents are described herein. In embodiments, the kit comprises a surface, and the anchoring reagent is provided on the surface. In embodiments, the kit comprises a surface, and the anchoring reagent is capable of being immobilized to the surface. In embodiments, the kit further comprises a reagent for immobilizing the anchoring reagent on the surface. Immobilization of anchoring reagents to surfaces are described herein. In embodiments, the anchoring reagent is directly immobilized onto the surface. In embodiments, the anchoring reagent is indirectly immobilized onto the surface, e.g., via secondary binding reagents. Secondary binding reagents, e.g., targeting agents and targeting agent complements, are further described herein.

In embodiments, the detection reagent comprises a nucleic acid probe, and the kit further comprises a template oligonucleotide and/or a polymerase. Template oligonucleotides and polymerases, e.g., for performing the PCR, LCR, SDA, 3SR, and/or isothermal amplification (such as, e.g., helicase-dependent amplification or RCA), are described herein. In embodiments, the kit further comprises a ligase, e.g., for ligating the template oligonucleotide. In embodiments, the template oligonucleotide is capable of hybridizing to the nucleic acid probe. In embodiments, the template oligonucleotide comprises a first region comprising a same sequence as the anchoring reagent. In embodiments, the kit further comprises a labeled probe. Labeled probes are further described herein. In embodiments, the labeled probe comprises a detection oligonucleotide, and the template oligonucleotide comprises a second region comprising a same sequence as the detection oligonucleotide. In embodiments, the labeled probe comprises a detectable label. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels. Detectable labels are further described herein.

In embodiments, the kit comprises a first detection reagent and a second detection reagent. First and second detection reagents are described herein. In embodiments, the first detection reagent and the second detection reagent are each independently an antibody, antigen, ligand, receptor, oligonucleotide, hapten, epitope, mimotope, or an aptamer. In embodiments, the first detection reagent and the second detection reagent are each independently an antibody or a variant thereof, including an antigen/epitope-binding portion thereof, an antibody fragment or derivative, an antibody analogue, an engineered antibody, or a substance that binds to antigens in a similar manner to antibodies. In embodiments, the first detection reagent and the second detection reagent each independently comprises at least one heavy or light chain complementarity determining region (CDR) of an antibody. In embodiments, the first detection reagent and the second detection reagent each independently comprises at least two CDRs from one or more antibodies. In embodiments, the first detection reagent and the second detection reagent are each independently an antibody or antigen-binding fragment thereof.

In embodiments, the first detection reagent binds tau. In embodiments, the first detection reagent is capable of binding to non-phosphorylated tau. In embodiments, the first detection reagent is capable of binding to pTau. In embodiments, the first detection reagent is capable of binding to both non-phosphorylated tau and pTau. In embodiments, the first detection reagent is capable of binding to pTau that is phosphorylated at T175, T181, T212, S214, T217, cis T231, trans T231, S293, S396, S610, or a combination thereof. In embodiments, the first detection reagent is capable of binding to pTau that is phosphorylated at T181.

In embodiments where the kit is for detecting pTau181, the second detection reagent specifically binds pTau181. In embodiments where the kit is for detecting pTau181, the second detection reagent binds pTau181 and does not bind non-phosphorylated tau. In embodiments where the kit is for detecting pTau181, the second detection reagent binds pTau181 and does not bind pTau that is not phosphorylated at amino acid position T181.

In embodiments where the kit is for detecting total tau, the second detection reagent is capable of binding non-phosphorylated tau and/or pTau, for example, pTau that is phosphorylated at any of amino acid positions T175, T181, T212, S214, T217, cis T231, trans T231, S293, S396, S610, or a combination thereof.

In embodiments, the first detection reagent and/or the second detection reagent comprises a detectable label. In embodiments, the first detection reagent and/or the second detection reagent is capable of being conjugated to a detectable label, and the kit further comprises the detectable label and/or a reagent for performing the conjugation. Conjugation of detectable labels to detection reagents are described herein. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels.

In embodiments, the first detection reagent comprises a first nucleic acid probe, and the second d detection reagent comprises a second nucleic acid probe. In embodiments, the first detection reagent is capable of being conjugated to a first second nucleic acid probe, the second detection reagent is capable of being conjugated to a second nucleic acid probe, and the kit further comprises the first nucleic acid, the second nucleic acid probe, and/or a reagent for performing the conjugation. Methods of conjugating nucleic acid probes to detection reagents are described herein. In embodiments, the first detection reagent is substantially non-conjugable with the second nucleic acid probe, and the second detection reagent is substantially non-conjugable with the first nucleic acid probe.

In embodiments, the first and second detection reagents comprise first and second nucleic acid probes, and the kit further comprises an anchoring reagent. Anchoring reagents are described herein. In embodiments, the kit comprises a surface, and the anchoring reagent is provided on the surface. In embodiments, the kit comprises a surface, and the anchoring reagent is capable of being immobilized to the surface. Immobilization of anchoring reagents to surfaces are described herein. In embodiments, the anchoring reagent is directly immobilized onto the surface. In embodiments, the anchoring reagent is indirectly immobilized onto the surface, e.g., via secondary binding reagents. Secondary binding reagents, e.g., targeting agents and targeting agent complements, are further described herein.

In embodiments, the first and second detection reagents comprise first and second nucleic acid probes, and the kit further comprises a template oligonucleotide and/or a polymerase. Template oligonucleotides and polymerases, e.g., for performing the PCR, LCR, SDA, 3SR, and/or isothermal amplification (such as, e.g., helicase-dependent amplification or RCA), are described herein. In embodiments, the kit further comprises a ligase, e.g., for ligating the template oligonucleotide. In embodiments, the template oligonucleotide is capable of hybridizing to the first and/or second nucleic acid probes. In embodiments, the template oligonucleotide comprises a first region comprising a same sequence as the anchoring reagent. In embodiments, the kit further comprises a labeled probe. Labeled probes are further described herein. In embodiments, the labeled probe comprises a detection oligonucleotide, and the template oligonucleotide comprises a second region comprising a same sequence as the detection oligonucleotide. In embodiments, the labeled probe comprises a detectable label. In embodiments, the detectable label is capable of being measured by light scattering, optical absorbance, fluorescence, chemiluminescence, electrochemiluminescence (ECL), bioluminescence, phosphorescence, radioactivity, magnetic field, or combinations thereof. In embodiments, the detectable label comprises one or more ECL labels. Detectable labels are further described herein.

In embodiments, the kits provided herein comprise a surface. In embodiments, a surface is provided separately from the components of the kit. In embodiments, the surface comprises a particle. In embodiments, the surface comprises a well of multi-well plate. In embodiments, the surface comprises a plurality of distinct binding domains. In embodiments, the surface comprises an electrode. In embodiments, the electrode is a carbon ink electrode. Surfaces and methods of immobilizing reagents thereon, e.g., capture and/or anchoring reagents, are further described herein.

In embodiments, one or more components of the kit are lyophilized. In embodiments where the kit comprises a detection reagent, the first capture reagent, the second capture reagent, and/or second detection reagent is lyophilized. In embodiments where the kit comprises a detection reagent, the first capture reagent, the second capture reagent, and/or the detection reagent is provided in solution. In embodiments where the kit comprises first and second detection reagents, the first capture reagent, the second capture reagent, the first detection reagent, and/or the second detection reagent is lyophilized. In embodiments where the kit comprises first and second detection reagents, the first capture reagent, the second capture reagent, the first detection reagent, and/or the second detection reagent is provided in solution.

In embodiments, the components of the kit, e.g., the capture and detection reagents and other components, are provided in separate containers, vials, or packages. In embodiments, the components of the kit are provided separately according to each component's optimal shipping and/or storage conditions.

In embodiments, the kit further comprises a polymerase (e.g., a polymerase described herein), a ligase (e.g., a ligase described herein), a calibration reagent, a buffer, a co-reactant, a blocking agent, a diluent, a stabilizing agent, an assay consumable, an electrode, or a combination thereof.

In embodiments, the kit further comprises a calibration reagent. In embodiments, the calibration reagent comprises a known quantity of non-phosphorylated tau, e.g., Tau441, or pTau, e.g., pTau181 or pTau217. In embodiments, the calibration reagent comprises a recombinant non-phosphorylated tau, e.g., Tau441, or a recombinant pTau, e.g., pTau181 or pTau217. In embodiments, the recombinant non-phosphorylated tau and/or the recombinant pTau is expressed in bacteria, e.g., E. coli, or in a mammalian system. In embodiments, the kit comprises multiple calibration reagents comprising a range of concentrations of non-phosphorylated tau or pTau, e.g., pTau181 or pTau217. In embodiments, the multiple calibration reagents comprise concentrations of non-phosphorylated tau or pTau, e.g., pTau181 or pTau217, near the upper and lower limits of quantitation for the method. In embodiments, the multiple calibration reagents span the entire dynamic range of the method. In embodiments, the calibration reagent is a positive control reagent. In embodiments, the calibration reagent is a negative control reagent. In embodiments, the positive or negative control reagent is used to provide a basis of comparison for the sample to be tested with the methods of the present invention. In embodiments, the calibration reagent is lyophilized. In embodiments, the calibration reagent is provided in solution.

In embodiments, the kit further comprises a buffer, e.g., an assay buffer, a reconstitution buffer, a storage buffer, a read buffer, or a combination thereof. In embodiments, the kit further comprises a co-reactant, e.g., for performing an ECL measurement. Exemplary ECL co-reactants are described, e.g., in WO 2020/142313 and PCT/US21/39835, filed Jun. 30, 2021.

In embodiments, the kit further comprises a blocking agent, e.g., to decrease non-specific binding by components other than tau to the capture and detection reagents described herein. Exemplary blocking agents include, but are not limited to, mBSA, sheared poly(A), polyBSA-I, mIgG, Tween, polyBSA-II, yeast RNA, mBSA+poly(a), and/or polyBSA+poly(A). In embodiments, the kit further comprises a diluent for one or more components of the kit. In embodiments, a kit comprising the components above includes stock concentrations of the components that are 5×, 10×, 20×, 30×, 40×, 50×, 60×, 70×, 80×, 90×, 100×, 125×, 150× or higher fold concentrations of a working concentration for the methods provided herein. In embodiments, the kit further comprises a stabilizing agent, e.g., for storage of one or more components of the kit.

In embodiments, the kit further comprises an assay consumable, e.g., assay modules, vials, tubes, liquid handling and transfer devices such as pipette tips, covers and seals, racks, labels, and the like. In embodiments, the kit further comprises an electrode, e.g., for performing an ECL measurement. In embodiments, the electrode is applied to the surface provided herein. In embodiments, the kit further comprises an assay instrument and/or instructions for carrying out the methods described herein.

All references cited herein, including patents, patent applications, papers, textbooks and the like, and the references cited therein, to the extent that they are not already, are hereby incorporated herein by reference in their entirety.

### SEQUENCES

amino acid sequence of human tau protein
SEQ ID NO: 1
MAEPRQEFEV MEDHAGTYGL GDRKDQGGYT MHQDQEGDTD
AGLKESPLQT PTEDGSEEPG SETSDAKSTP TAEDVTAPLV
DEGAPGKQAA AQPHTEIPEG TTAEEAGIGD TPSLEDEAAG
HVTQARMVSK SKDGTGSDDK KAKGADGKTK IATPRGAAPP
GQKGQANATR IPAKTPPAPK TPPSSGEPPK SGDRSGYSSP
GSPGTPGSRS RTPSLPTPPT REPKKVAVVR TPPKSPSSAK
SRLQTAPVPM PDLKNVKSKI GSTENLKHQP GGGKVQIINK
KLDLSNVQSK CGSKDNIKHV PGGGSVQIVY KPVDLSKVTS
KCGSLGNIHH KPGGGQVEVK SEKLDFKDRV QSKIGSLDNI
THVPGGGNKK IETHKLTFRE NAKAKTDHGA EIVYKSPVVS
GDTSPRHLSN VSSTGSIDMV DSPQLATLAD EVSASLAKQG
L

### EXAMPLES

### Example 1. PTau181 and Total Tau Assays for Cognitive Decline Assessment

Plasma samples from 455 individuals were subjected to 59 different biomarker assays. The individuals were grouped as follows based on CDR® Dementia Staging Instrument, MMSE score, and MoCA scores:
Normal (high contrast);
Normal, decline;
Subjective Cognitive Complaints (SCC), stable;
Subjective Cognitive Complaints (SCC), decline;
Mild Cognitive Impairment (MCI), stable;
Mild Cognitive Impairment (MCI), decline;
Mild Cognitive Impairment (MCI), AD;
Mild Cognitive Impairment (MCI), Non-AD;
Neurodegenerative, Non-AD;
Dementia, Non-AD;
AD (Path);
AD;
AD (high contrast).

“High contrast” indicates individuals who were classified based on CSF/PET data. “Path” indicates individuals whose diagnoses were pathology verified. “Stable” refers to individuals whose diagnoses or symptoms remain do not progress to any further cognitive impairment during the duration of the tracked study. “Decline” refers to individuals diagnoses or symptoms worsen, e.g., from cognitively normal to MCI or dementia, or from MCI to dementia, during the duration of the tracked study.

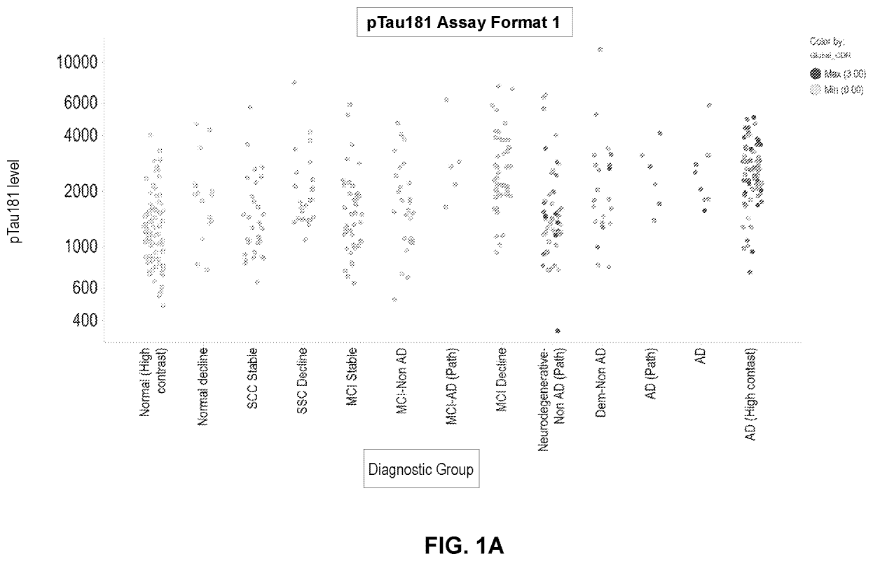

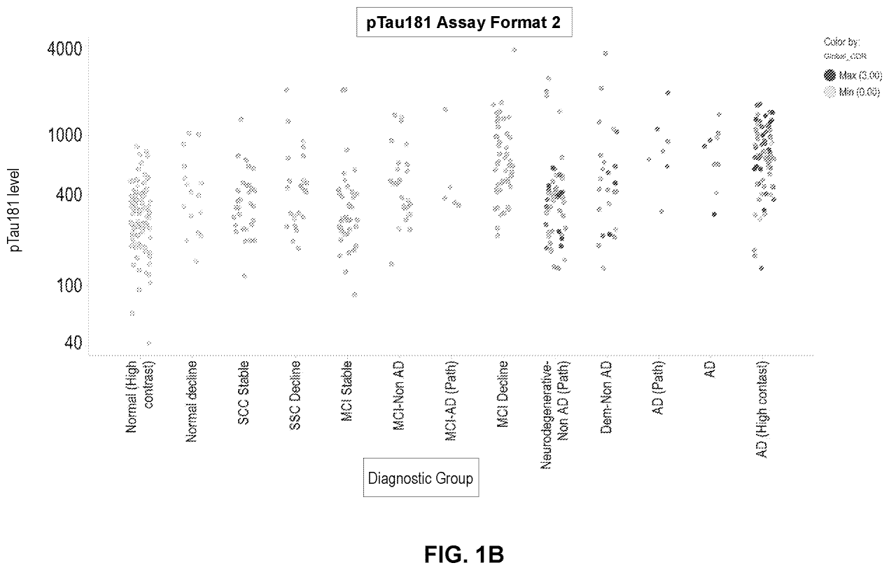

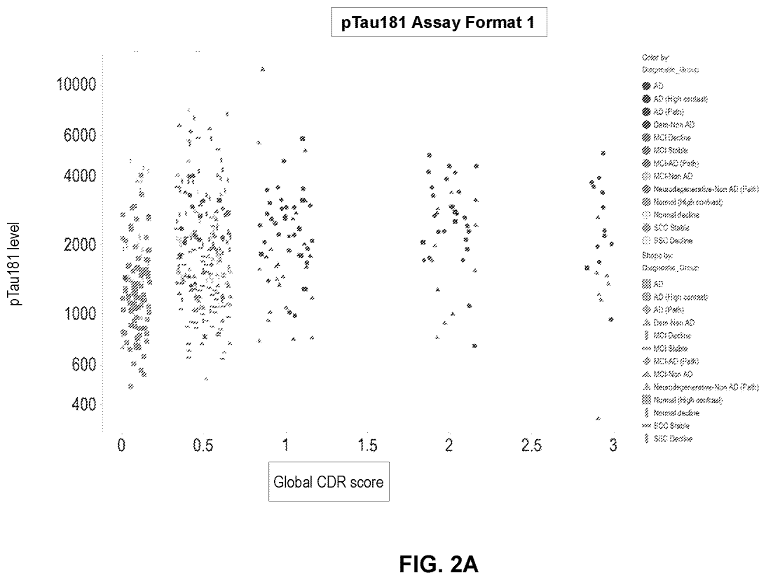

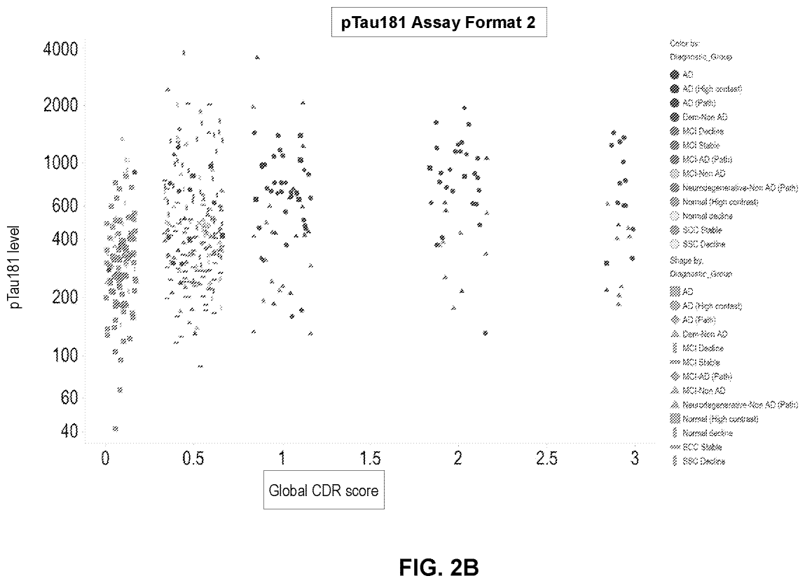

The biomarker pTau181 showed significant distinction between diagnostic groups and was highly significantly associated with cognition scores. As shown in FIGS. 1A and 1B, pTau181 concentrations were significantly lower for the Normal group than for all disease groups. pTau181 concentrations were also significantly lower for groups that remained stable than for groups that later had cognitive decline. Further, FIGS. 2A and 2B show that pTau181 concentrations were significantly associated with cognition scores. In FIGS. 1A and 2A, the pTau181 levels were measured with an assay using first and second capture reagents (“dual-capture assay”) and a detection reagent linked to a nucleic acid probe, as described herein (pTau181 Assay Format 1). In FIGS. 1B and 2B, the pTau181 levels were measured using a dual-capture assay with a first detection reagent linked to a first nucleic acid probe, and a second detection reagent linked to a second nucleic acid probe, as described herein (pTau181 Assay Format 2).

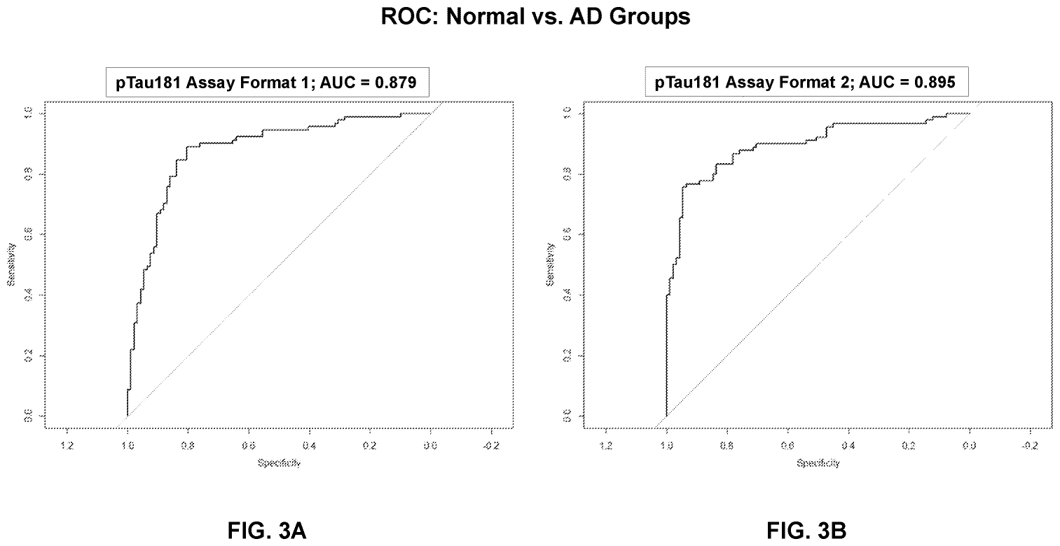

FIGS. 3A and 3B show receiver operating characteristic (ROC) curves for using pTau181 to distinguish between individuals in the Normal (high contrast) group versus the AD, AD (Path), and AD (high contrast) groups (collectively “AD groups”). The pTau181 levels were measured using pTau181 Assay Format 1 or pTau181 Assay Format 2 described herein, and the AUC values for the two assay formats were 0.879 and 0.895, respectively, indicating a high degree of discrimination between normal and AD groups.

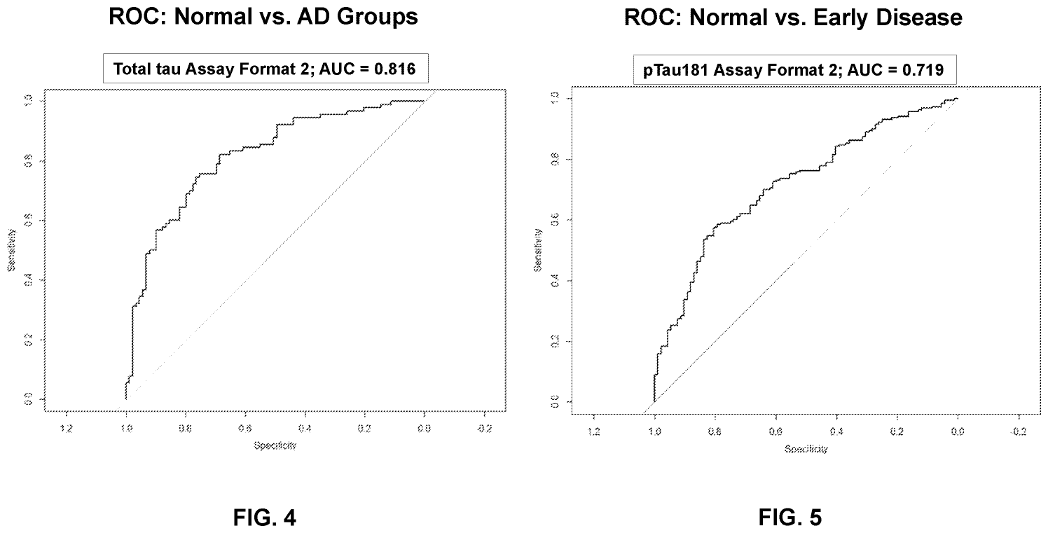

Total tau was also shown to distinguish between the Normal (high contrast) group and AD groups, as shown in FIG. 4. Total tau was measured using a dual-capture assay with a detection reagent comprising a detectable label, as described herein (total tau Assay Format 2).

FIG. 5 shows that pTau181 distinguished between the Normal (high contrast) group and the following groups with SCC or MCI, which were indicative of early disease: Normal, decline; SCC, stable; SCC, decline; MCI, stable; MCI, decline; MCI, AD; and MCI, Non-AD (collectively “early disease group”). Levels of pTau181 were measured using pTau181 Assay Format 2, providing an AUC of 0.719.

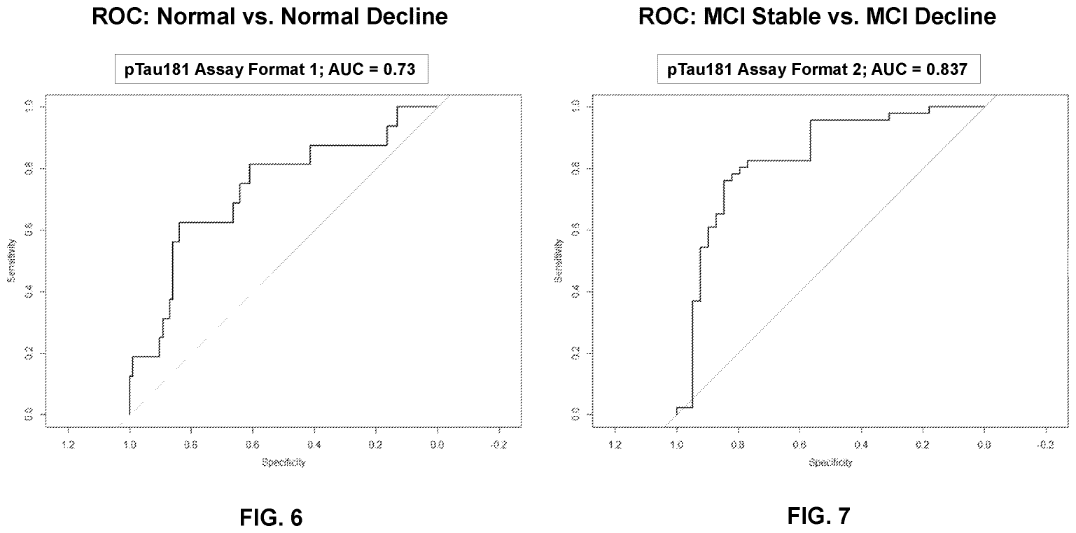

FIG. 6 shows that pTau181 also distinguished between the Normal (high contrast) group (i.e., cognitively normal individuals who remain stable) and the Normal, decline group (i.e., cognitively normal individuals who later decline). Levels of pTau181 were measured using pTau181 Assay Format 1, providing an AUC of 0.73.

FIG. 7 shows that pTau181 further distinguished between the MCI, stable group (i.e., individuals with MCI who remain stable) and the MCI, decline group (i.e., individuals with MCI who later decline). Levels of pTau181 were measured using pTau181 Assay Format 2, providing an AUC of 0.837.

Thus, the results show that pTau181 effectively discriminates between normal versus early disease groups, and stable versus decline groups.

### Example 2. Further Biomarker Study

In this Example, two additional cohorts were assessed using biomarker assays described in embodiments herein.

### Biomarker Assays

Plasma samples from individuals were collected in tubes containing EDTA, centrifuged at 2000×g for 5 minutes, frozen in cryovials within 4 hours of collection, and stored at −80° C. until use. Biomarker assays according to embodiments herein were conducted to measure levels of pTau181 and total tau. Recombinant phosphorylated tau, expressed in a mammalian system and confirmed by mass spectrometry to display phosphorylation at T181, was used as calibrator for the pTau181 assay. Recombinant Tau441, expressed in E. coli, was used as calibrator for the total tau assay. Lowest limit of detection (LLOD) was defined as the concentration that provides a signal 2.5 standard deviations above the mean of the blank. LLOQ was defined as the lowest concentration with a CV<20% and a recovery between 80-120%. Plasma samples were measured as single replicates using 25 μL of undiluted plasma for the pTau181 assay, or 25 μL of 5-fold diluted plasma for the total tau assay. Reported concentrations of total tau was corrected for the 5-fold sample dilution.

Biomarker concentrations were log transformed to satisfy assumptions of distribution normality. Values under LLOQ were assigned the lowest quantifiable value of the assay. All reported p-values were adjusted for multiple hypothesis testing using the Benjamini-Hochberg method unless otherwise specified. Differences between diagnostic groups were evaluated using ANOVA adjusting for age, sex, and the biomarker in question followed by Tukey's Honest Significant Difference as post-hoc test. Subgroup analyses between different clinical subsets were performed using logistic regression predicting the subgroup in terms of age, sex, and the relevant biomarker. To assess classification utility of the biomarkers, area under the curve (AUC) values were computed using logistic regression models as described in Robin et al., BMC Bioinformatics 12:77 (2011). Assay performance is shown in Table 2.

TABLE 2
Biomarker Assay Performance

| | LLOD | LLOQ | ULOQ | Median conc (IQR) | CV for QC |
|---|---|---|---|---|---|
| | (pg/mL) | (pg/mL) | (pg/mL) | (pg/mL) | samples |
| pTau181 | 0.08 | 0.46 | 990 | 1.7 (1.2-2.6) | 8% |
| total tau | 0.07 | 0.63 | 2,000 | 10.2 (8.2-13.4) | 4-7% |

LLOD: lower level of detection; L/ULOQ: lower/upper level of quantification; IQR = interquartile range; CV = coefficient of variation; QC = quality control.

### Cohort A

Cohort A included 238 individuals, divided among three groups: AD as determined by autopsy (n=75) or molecular biomarkers (CSF or amyloid PET; n=20), other neurodegenerative diseases without AD neuropathological changes on autopsy (ONDs, n=53), and healthy individuals with normal neuropsychological testing scores and no subjective cognitive symptoms (HC, n=90). It was found that pTau181 and total tau biomarker levels increased with age in the HC group. Additionally, a significant effect of sex for pTau181 in the HC group, with males having higher pTau181 levels than females (p<0.002). Thus, all subsequent analyses were controlled for age and sex.

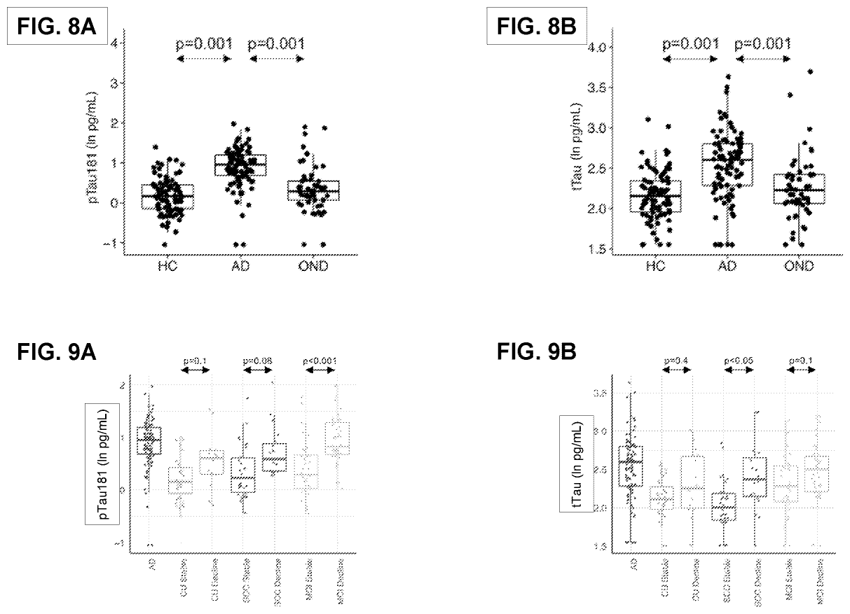

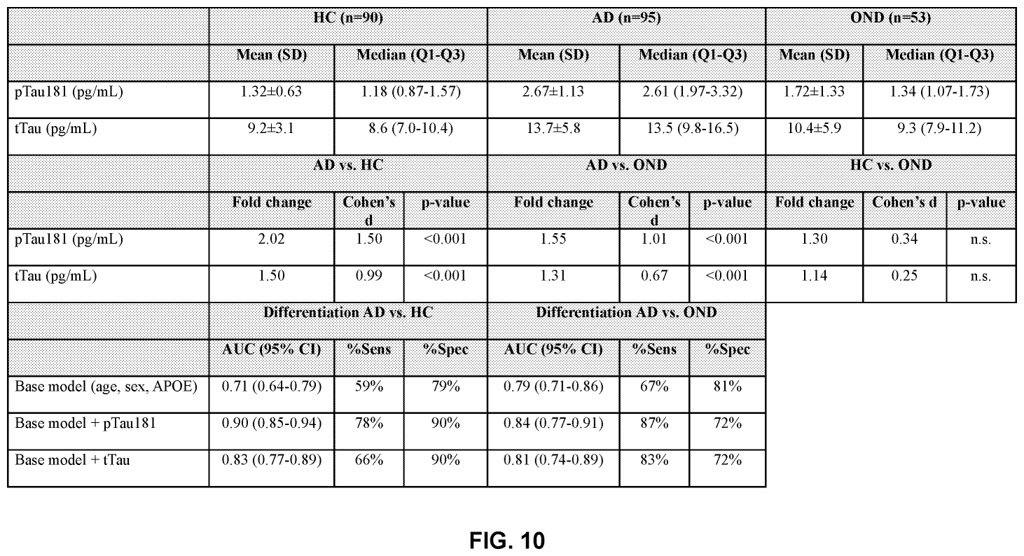

Between-group differences in plasma biomarker concentrations were determined using ANOVA followed by Tukey's post-hoc test. This analysis demonstrated that individuals with AD had roughly 2-fold higher plasma concentrations of pTau181 compared to HC, while total tau concentrations were 1.5-fold higher in individuals with AD (p<0.001 for all comparisons). A logistic regression model where biomarker levels were residualized on age to control for any age effects showed an excellent ability for pTau181 to discriminate between individuals with AD and HC individuals with an AUC of 0.88, with 82% sensitivity and 88% specificity at an optimal threshold defined by the Youden index. Individuals with AD also had roughly 1.3-1.5 fold higher plasma pTau181 and total tau concentrations than ONDs (p<0.001). Results are summarized in FIG. 8A (pTau181), FIG. 8B (total tau), and FIG. 10.

A further analysis was performed with a logistic regression base model with age, sex, and APOE status of the individuals. APOE status of the individuals was determined by APOE genotyping. The AUC for differentiating between individuals with AD and HC individuals in the base model was 0.71. Adding pTau181 to the base model increased the AUC for differentiating between individuals with AD and HC individuals to 0.90 (p<0.001) with 78% sensitivity and 90% specificity. Further, adding pTau181 to the base model increased the AUCs for differentiating individuals with AD and individuals with ONDs from 0.79 to 0.84. See FIG. 10.

### Cohort B

Cohort B included: a) 85 individuals who had a baseline diagnosis of MCI due to probable AD, classified into two groups: MCI-decline (n=47) and MCI-stable (n=38); b) 49 participants who had a baseline clinical status of subjective cognitive complaints (SCC), classified into two groups: SCC-decline (n=19) and SCC-stable (n=30); and c) 53 participants who had no cognitive complaints (cognitively unimpaired, CU) and classified into two groups: CU-decline (n=14) and CU-stable (n=39). Individuals were classified as “decline” or “stable” based on their scores on the Clinical Dementia Rating (CDR) scale over the course of four years with at least five annual follow-up visits. Further, individuals with SCC were only considered as SCC-decline individuals if they had at least two consecutive follow up visits in which their consensus diagnosis was MCI and their global CDR was 0.5, without subsequent return to SCC status. 15 MCI-decline, 2 MCI-stable, 1 SCC-stable, 2 CU-decline, and all CU-stable individuals were also included in the cross-sectional diagnostic Cohort A. There were no differences in age or sex between MCI-stable and MCI-decline individuals, while the CU-decline and SCC-decline groups tended to be older than their stable counterparts. Age and sex were therefore included as covariates in the analysis.

MCI-decline individuals had 1.7-fold higher levels of pTau181 compared with MCI-stable individuals (p<0.0005). There were no differences in pTau181 levels between MCI-decline individuals (mean+SD: 2.88+1.44 pg/mL) and individuals with AD (mean±SD:2.67±1.13 pg/mL). pTau181 showed excellent ability to discriminate between MCI-decline and MCI-stable individuals with an AUC for pTau181 of 0.81 with 85% sensitivity and 68% specificity.

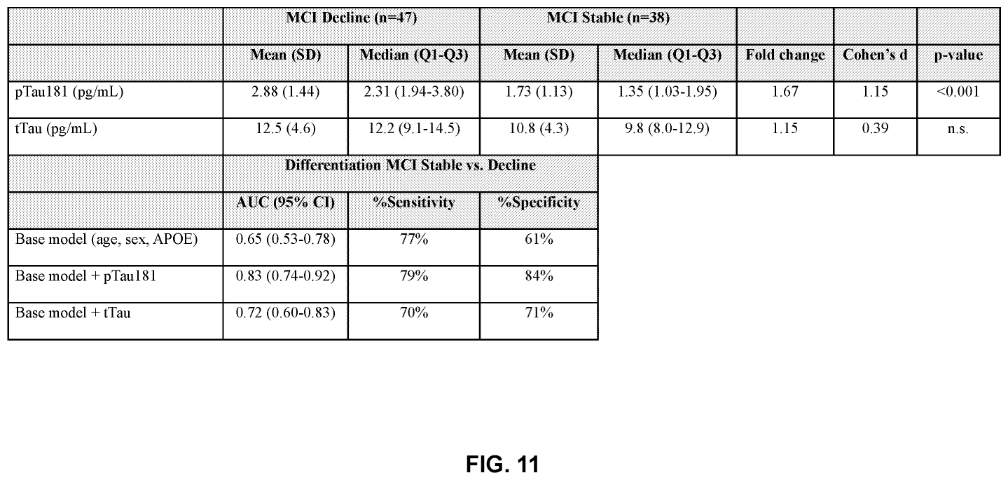

A further analysis was performed with a logistic regression base model with age, sex, and APOE status of the individuals as described above for Cohort A. Adding pTau181 increased the ability to discriminate between MCI-decline and MCI-stable individuals from an AUC of 0.65 (base model) to an AUC of 0.83 (base model+pTau181). See FIG. 11.

SCC-decline individuals had 1.5-fold higher levels of pTau181 and total tau compared with SCC-stable individuals (total tau: p<0.07). The relatively high p-value is attributed to relatively small sample size and common diagnostic atypicality.

There were no statistically significant differences in baseline biomarker concentrations between CU-stable and CU-decline individuals. However, the number of CU-decline individuals was small (n=14). Results are summarized in FIG. 9A (pTau181) and FIG. 9B (total tau).

### Example 3. Alzheimer's vs. Non-Alzheimer's Dementia

An exemplary method for distinguishing between Alzheimer's disease and non-Alzheimer's dementia in an individual is provided.

The individual's pTau181 and neurofilament light chain protein (NfL) levels are measured. Levels of pTau181 are measured using the methods described herein. Levels of NfL are measured using analogous assays as described herein and/or using methods known to one of ordinary skill in the art. Individuals with levels of pTau181 that are within a normalized concentration and with levels of NfL that are elevated as compared to a normalized concentration are determined with high likelihood of having non-Alzheimer's dementia. The normalized concentration is determined in a cohort to which the individual belongs, e.g., based on the individual's gender, age group, preexisting health condition(s), family history, genetic factors (e.g., APOE status), levels of other biomarkers, or combinations thereof.

### Example 4. Development of a pTau217 Assay

Preliminary testing showed that pTau217 was elevated in serum and plasma from AD patients relative to controls.

The assay was conducted using a capture reagent specific to pT217 and a detection reagent specific to tau, referred to as “the pTau217 assay” below.

The samples were obtained from a “high contrast” AD group that included 7 MCI and 11 dementia patients; and a control group that included 10 normal volunteer subjects, 5 subjects with normal pressure hydrocephalus, 2 subjects with polyneuropathy, and 1 subject with unknown status. The subjects' demographics are summarized in Table 1 below.

TABLE 1

| | Age | Sex |
|---|---|---|
| Group | Mean (SD), Range | M/F |
| AD | 71.3 (8.2), 57-90 | 10/8 |
| Control | 70.6 (8.9), 57-82 | 11/7 |

As shown in Table 1, the groups were well matched based on age and gender.

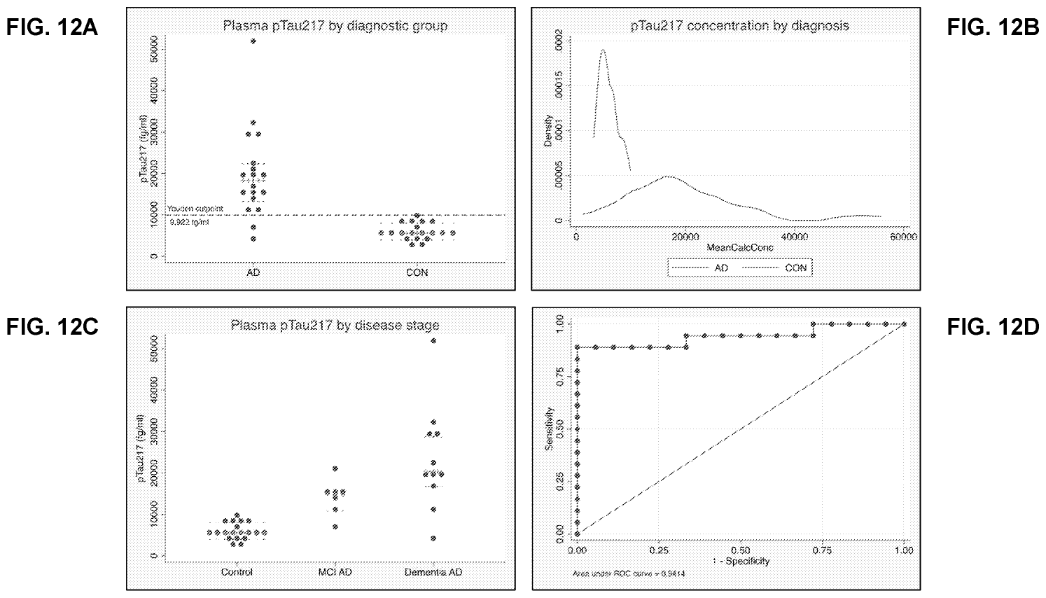

The plasma samples from the subjects were tested with the pTau217 assay, and results of pTau217 levels as grouped by diagnostic group, diagnosis, and disease stage are shown in FIGS. 12A-12C, respectively. FIG. 12D shows that the pTau217 assay had an AUC of 0.94, which is comparable to the AUC of the pTau181 assay. Table 2 shows a summary of the mean pTau217 levels and fold-change between the AD vs. control groups. The pTau217 levels from AD patients had an estimated 3.3-fold increase as compared with the control group, which is larger than the ˜2-fold increase previously measured for pTau181.

TABLE 2

| | pTau217 | Fold | |
|---|---|---|---|
| Group | Mean (SD) | Change | t-test |
| AD | 19.7 (11.0) | 3.3 | p < 0.00006 |
| Control | 6.0 (2.0) | | |

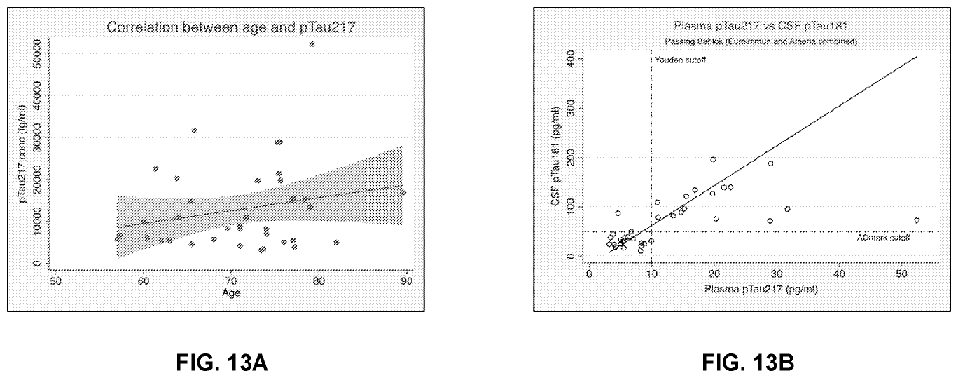

FIG. 13A shows that pTau217 levels correlate with age. FIG. 13B shows that pTau181 and pTau217 levels generally correlate, and the pTau217 assay can achieve 89% sensitivity with 100% specificity with a cut-off value of 9.9 pg/mL between the AD and control groups.

1. A method of detecting phosphorylated tau (pTau) in a sample, wherein the pTau is phosphorylated at amino acid position T181 (pTau181) or amino acid position T217 (pTau217), comprising:
a) contacting the sample with: (i) a first capture reagent that binds tau; (ii) a second capture reagent that binds tau; and (iii) a detection reagent that binds pTau181 or pTau217,
thereby forming a complex comprising the first and second capture reagents, pTau181 or pTau217, and the detection reagent; and
b) detecting the complex, thereby detecting the pTau181 or pTau217,
wherein the sample is from a subject with subjective cognitive complaints (SCC), diagnosed with mild cognitive impairment (MCI), or at risk of developing MCI.

2. A method of detecting phosphorylated tau (pTau) in a sample, wherein the pTau is phosphorylated at amino acid position T181 (pTau181) or amino acid position T217 (pTau217), comprising:
a) contacting the sample with: (i) a first capture reagent that binds tau; (ii) a second capture reagent that binds tau; and (iii) a detection reagent that binds pTau181 or pTau217,
thereby forming a complex comprising the first and second capture reagents, pTau181 or pTau217, and the detection reagent; and
b) detecting the complex, thereby detecting the pTau181 or pTau217,
wherein the sample comprises less than 75% pTau181 as compared to a normalized pTau181 concentration, and/or wherein the sample comprises less than 75% pTau217 as compared to a normalized pTau217 concentration.

3. A method of detecting phosphorylated tau (pTau) in a sample, wherein the pTau is phosphorylated at amino acid position T181 (pTau181) or amino acid position T217 (pTau217), comprising:
a) contacting the sample with: (i) a first capture reagent that binds tau; (ii) a second capture reagent that binds tau; and (iii) a detection reagent that binds pTau181 or pTau217,
thereby forming a complex comprising the first and second capture reagents, pTau181 or pTau217, and the detection reagent; and
b) detecting the complex, thereby detecting the pTau181 or pTau217,
wherein the sample comprises greater than 120% pTau181 as compared to a normalized pTau181 concentration, and/or wherein the sample comprises greater than 120% pTau217 as compared to a normalized pTau217 concentration.

4. A method of detecting pTau181 in a sample, comprising:
a) contacting the sample with: (i) a first capture reagent that binds tau; (ii) a second capture reagent that binds tau; (iii) a first detection reagent that binds tau; and (iv) a second detection reagent that binds pTau181,
thereby forming a complex comprising the first and second capture reagents, pTau181, and the first and second detection reagents; and
b) detecting the complex, thereby detecting pTau181,
wherein the sample is from a subject with subjective cognitive complaints (SCC), diagnosed with mild cognitive impairment (MCI), or at risk of developing MCI.

5. A method of detecting pTau181 in a sample, comprising:
a) contacting the sample with: (i) a first capture reagent that binds tau; (ii) a second capture reagent that binds tau; (iii) a first detection reagent that binds tau; and (iv) a second detection reagent that binds pTau181,
thereby forming a complex comprising the first and second capture reagents, pTau181, and the first and second detection reagents;
b) detecting the complex, thereby detecting pTau181,
wherein the sample comprises less than 75% pTau181 as compared to a normalized pTau181 concentration.

6. A method of detecting pTau181 in a sample, comprising:
a) contacting the sample with: (i) a first capture reagent that binds tau; (ii) a second capture reagent that binds tau; (iii) a first detection reagent that binds tau; and (iv) a second detection reagent that binds pTau181,
thereby forming a complex comprising the first and second capture reagents, pTau181, and the first and second detection reagents;
b) detecting the complex, thereby detecting pTau181,
wherein the sample comprises greater than 120% pTau181 as compared to a normalized pTau181 concentration.

7. The method of any one of claims 1 to 6, wherein the complex is bound to a surface prior to the detecting.

8. The method of any one of claims 1 to 3, wherein the detection reagent comprises a detectable label, and the detecting comprises measuring the amount of the detectable label.

9. The method of any one of claims 1 to 3, wherein the detection reagent comprises a nucleic acid probe, the complex is bound to a surface prior to the detecting, the surface further comprises an anchoring reagent, and the detecting comprises:
extending the nucleic acid probe to form an extended sequence comprising an anchoring region that binds to the anchoring reagent;
binding the extended sequence to the anchoring reagent; and
measuring the amount of extended sequence bound to the surface.

10. The method of any one of claims 4 to 6, wherein the first detection reagent comprises a first nucleic acid probe, the second detection reagent comprises a second nucleic acid probe, the complex is bound to a surface prior to the detecting, the surface further comprises an anchoring reagent, and the detecting comprises:
extending the second nucleic acid probe to form an extended sequence comprising an anchoring region that binds to the anchoring reagent;
binding the extended sequence to the anchoring reagent; and
measuring the amount of extended sequence bound to the surface.

11. The method of any one of claims 1 to 3, claim 8, or claim 9, wherein the detection reagent that binds pTau181 does not bind non-phosphorylated tau.

12. The method of any one of claims 4 to 6 or claim 10, wherein the first detection reagent that binds tau is capable of binding to both non-phosphorylated tau and phosphorylated tau.

13. The method of any one of claims 4 to 6, claim 10, or claim 11, wherein the second detection reagent that binds pTau181 does not bind non-phosphorylated tau.

14. The method of any one of claims 1 to 13, wherein the first and second capture reagents that bind tau are capable of binding to both non-phosphorylated tau and phosphorylated tau

15. The method of any one of claims 1 to 14, wherein each capture reagent and each detection reagent comprises an antibody or antigen-binding fragment thereof, antigen, ligand, receptor, oligonucleotide, hapten, epitope, mimotope, or an aptamer.

16. The method of any one of claims 1 to 15, wherein each capture reagent and each detection reagent comprises an antibody or antigen-binding fragment thereof.

17. The method of claim 9, claim 10, or any one of claims 11 to 16, wherein the extending comprises polymerase chain reaction (PCR), ligase chain reaction (LCR), strand displacement amplification (SDA), self-sustained synthetic reaction (3SR), isothermal amplification, or combination thereof.

18. The method of claim 9 or any one of claims 11 to 17, wherein the extending comprises binding the nucleic acid probe to a template oligonucleotide, forming a circular template oligonucleotide, and extending the nucleic acid probe by rolling circle amplification.

19. The method of any one of claims 10 to 17, wherein the extending comprises binding the first and second nucleic acid probes to a template oligonucleotide, forming a circular template oligonucleotide, and extending the nucleic acid probe by rolling circle amplification.

20. The method of any one of claims 9 to 19, wherein the anchoring reagent comprises an anchoring oligonucleotide, and the extended sequence comprises an anchoring oligonucleotide complement that is complementary to the anchoring oligonucleotide.

21. The method of any one of claims 9 to 20, wherein the extended sequence comprises a detection oligonucleotide complement.

22. The method of claim 21, wherein the measuring comprises:
contacting the extended sequence with a labeled probe comprising: (i) a detection oligonucleotide that is complementary to the detection oligonucleotide complement; and (ii) a detectable label; and
measuring the amount of detectable label bound to the surface.

23. The method of claim 8 or claim 22, wherein the detectable label is capable of being measured by a measurement of light scattering, optical absorbance, fluorescence, chemiluminescence, electrochemiluminescence (ECL), bioluminescence, phosphorescence, radioactivity, magnetic field, or combinations thereof.

24. The method of claim 23, wherein the detectable label is an ECL label, and the measuring comprises measuring an ECL signal.

25. The method of any one of claims 1 to 24, wherein the surface comprises a particle.

26. The method of any one of claims 1 to 24, wherein the surface comprises a well of a multi-well plate.

27. The method of any one of claims 1 to 26, wherein the surface comprises an electrode, and the detecting further comprises applying a potential to the electrode and measuring electrochemiluminescence.

28. The method of claim 25, further comprising collecting the particle on an electrode, and the detecting further comprises applying a potential to the electrode and measuring electrochemiluminescence.

29. The method of any one of claims 9 to 28, wherein the surface comprises a plurality of distinct binding domains, and the first capture reagent, the second capture reagent, and the anchoring reagent are located on two or more distinct binding domains on the surface.

30. The method of any one of claims 9 to 28, wherein the surface comprises a plurality of distinct binding domains, and the first capture reagent, the second capture reagent, and the anchoring reagent are located on the same binding domains on the surface.

31. A method of determining if a cognitively normal subject is likely to experience cognitive decline within 10 years, the method comprising:
a) obtaining a measurement of pTau181, pTau217, and/or total tau levels of the subject; and
b) determining if the subject is likely to experience cognitive decline based on the measurement of pTau181, pTau217, and/or total tau levels.

32. A method of preventing, reducing, or delaying cognitive decline in a cognitively normal subject, the method comprising:
a) obtaining a measurement of pTau181, pTau217, and/or total tau levels of the subject;
b) identifying the subject as being likely to experience future cognitive decline based on the measurement of pTau181, pTau217, and/or total tau levels; and
c) administering a regimen to the subject to prevent, reduce, or delay cognitive decline.

33. A method of determining if a subject with subjective cognitive complaints is likely to experience cognitive decline within 10 years, the method comprising:
a) obtaining a measurement of pTau181, pTau217, and/or total tau levels of the subject; and
b) determining if the subject is likely to experience cognitive decline based on the measurement of pTau181, pTau217, and/or total tau levels.

34. A method of preventing, reducing, or delaying cognitive decline in a subject with subjective cognitive complaints, the method comprising:
a) obtaining a measurement of pTau181, pTau217, and/or total tau levels of the subject;
b) identifying the subject as being likely to experience future cognitive decline based on the measurement of pTau181, pTau217, and/or total tau levels; and
c) administering a regimen to the subject to prevent, reduce, or delay cognitive decline.

35. A method of determining if a subject with mild cognitive impairment (MCI) is likely to experience further cognitive decline within 10 years, the method comprising:
a) obtaining a measurement of pTau181, pTau217, and/or total tau levels of the subject; and
b) determining if the subject is likely to experience further cognitive decline based on the measurement of pTau181, pTau217, and/or total tau levels.

36. A method of preventing, reducing, or delaying further cognitive decline in a subject with mild cognitive impairment (MCI), the method comprising:
a) obtaining a measurement of pTau181, pTau217, and/or total tau levels of the subject;
b) identifying the subject as being likely to experience further cognitive decline based on the measurement of pTau181, pTau217, and/or total tau levels; and
c) administering a regimen to the subject to prevent, reduce, or delay further cognitive decline.

37. A method of determining eligibility of a subject to participate in a clinical trial of a therapeutic drug for preventing or delaying Alzheimer's disease, the method comprising:
a) obtaining a measurement of pTau181, pTau217, and/or total tau levels of the subject;
b) determining the eligibility of the subject for the clinical trial based on the measurement of pTau181, pTau217, and/or total tau levels;
wherein the subject has been diagnosed with dementia or mild cognitive impairment, or
wherein the subject has subjective cognitive complaints, or
wherein the subject does not have any cognitive impairment.

38. A method of conducting a clinical trial of a therapeutic drug or intervention for Alzheimer's disease, the method comprising:
a) obtaining a measurement of pTau181, pTau217, and/or total tau levels of a subject;
b) determining eligibility of the subject for the clinical trial based on the measurement of pTau181, pTau217, and/or total tau levels; and
c) administering the therapeutic drug to the subject.

39. A method of distinguishing a subject afflicted with Alzheimer's disease from an individual afflicted with non-Alzheimer's dementia, the method comprising:
a) obtaining a measurement of pTau181, pTau217, and/or total tau levels of the subject; and
b) identifying, based on the measurement of pTau181, pTau217, and/or total tau levels, the subject as (i) afflicted with Alzheimer's disease or (ii) afflicted with non-Alzheimer's dementia.

40. A method of treating Alzheimer's disease in a subject in need thereof, the method comprising:
a) obtaining a measurement of pTau181, pTau217, and/or total tau levels of the subject, wherein the measurement is obtained prior to administration of a treatment for Alzheimer's disease,
b) determining, based on the measurement of pTau181, pTau217, and/or total tau levels, that the subject is afflicted with Alzheimer's disease, and
c) administering a treatment regimen for Alzheimer's disease to the subject.

41. A method of monitoring response to treatment for Alzheimer's disease in a subject, the method comprising:
a) obtaining a first measurement of pTau181, pTau217, and/or total tau levels of the subject, wherein the first measurement is obtained prior to administration of a treatment regimen for Alzheimer's disease,
b) obtaining a second measurement of pTau181, pTau217, and/or total tau levels of the subject at one or more time points after administration of the treatment regimen for Alzheimer's disease has been initiated,
c) determining, based on the first and second measurements of pTau181, pTau217, and/or total tau levels, that the subject is responding positively to the Alzheimer's treatment regimen, and
d) continuing to administer the treatment regimen for Alzheimer's disease to the subject.

42. The method of any one of claims 31 to 41, wherein the measurement of pTau181 or pTau217 level is measured by a method according to any one of claims 1 to 30.

43. The method of any one of claims 31 to 41, wherein the measurement of total tau level is measured by a method comprising:
a) contacting the sample with: (i) a first capture reagent that binds tau; (ii) a second capture reagent that binds tau; and (iii) a detection reagent that binds tau, thereby forming a complex comprising the first and second capture reagents, tau, and the detection reagent; and
b) detecting the complex, thereby detecting total tau.

44. The method of any one of claims 31 to 41, wherein the total tau level is measured by a method comprising:
a) contacting the sample with: (i) a first capture reagent that binds tau; (ii) a second capture reagent that binds tau; (iii) a first detection reagent that binds tau; and (iv) a second detection reagent that binds tau,
thereby forming a complex comprising the first and second capture reagents, tau, and the first and second detection reagents; and
b) detecting the complex, thereby detecting total tau.

45. The method of claim 43 or 44, wherein the complex is bound to a surface prior to the detecting.

46. The method of claim 43, wherein the detection reagent comprises a detectable label, and the detecting comprises measuring the amount of the detectable label.

47. The method of claim 43, wherein the detection reagent comprises a nucleic acid probe, the complex is bound to a surface prior to the detecting, the surface further comprises an anchoring reagent, and the detecting comprises:
extending the nucleic acid probe to form an extended sequence comprising an anchoring region that binds to the anchoring reagent;
binding the extended sequence to the anchoring reagent; and
measuring the amount of extended sequence bound to the surface.

48. The method of claim 44, wherein the first detection reagent comprises a first nucleic acid probe, the second detection reagent comprises a second nucleic acid probe, the complex is bound to a surface prior to the detecting, the surface further comprises an anchoring reagent, and the detecting comprises:
extending the second nucleic acid probe to form an extended sequence comprising an anchoring region that binds to the anchoring reagent;
binding the extended sequence to the anchoring reagent; and
measuring the amount of extended sequence bound to the surface.

49. The method of any one of claims 1 to 48, wherein the sample comprises whole blood, blood serum plasma, cerebrospinal fluid, urine, saliva, or an extraction or purification therefrom, or dilution thereof.

50. The method of claim 49, wherein the sample comprises plasma.

51. A kit for detecting pTau181 and/or pTau217 comprising, in one or more vials, containers, or compartments:
a) a first capture reagent that binds tau;
b) a second capture reagent that binds tau;
c) a detection reagent that binds pTau181 and/or a detection reagent that binds pTau217; and
d) optionally a surface,
wherein the first and second capture reagents are provided on the surface or are capable of binding to the surface.

52. A kit for detecting pTau181 comprising, in one or more vials, containers, or compartments:
a) a first capture reagent that binds tau;
b) a second capture reagent that binds tau;
c) a first detection reagent that binds tau;
d) a second detection reagent that binds pTau181; and
e) optionally a surface,
wherein the first and second capture reagents are provided on the surface or are capable of binding to the surface.

53. The kit of claim 51, wherein the detection reagent comprises a detectable label or is capable of being conjugated to a detectable label.

54. The kit of claim 51, wherein the detection reagent comprises a nucleic acid probe or is capable of being conjugated to a nucleic acid probe.

55. The kit of claim 52, wherein the first detection reagent comprises a first nucleic acid probe or is capable of being conjugated to a first nucleic acid probe, and the second detection reagent comprises a second nucleic acid probe or is capable of being conjugated to a second nucleic acid probe.

56. The kit of claim 54 or 55, further comprising an anchoring reagent, wherein the anchoring reagent is provided on the surface or is capable of binding to the surface.

57. The kit of any one of claim 51, 53, 54, or 56, wherein the detection reagent that binds pTau181 does not bind non-phosphorylated tau.

58. The kit of any one of claim 52, 55, or 56, wherein the first detection reagent that binds tau is capable of binding to both non-phosphorylated tau and phosphorylated tau.

59. The kit of any one of claim 52, 55, 56, or 58, wherein the second detection reagent that binds pTau181 does not bind non-phosphorylated tau.

60. The kit of any one of claims 51 to 59, wherein the first and second capture reagents that bind tau are capable of binding to both non-phosphorylated tau and phosphorylated tau.

61. The kit of any one of claims 51 to 60, wherein each capture reagent and each detection reagent comprises an antibody or antigen-binding fragment thereof, antigen, ligand, receptor, oligonucleotide, hapten, epitope, mimotope, or an aptamer.

62. The kit of any one of claims 51 to 61, wherein each capture reagent and each detection reagent comprises an antibody or antigen-binding fragment thereof.

63. The kit of any one of claims 54 to 62, further comprising a labeled probe that comprises (i) a detection oligonucleotide and (ii) a detectable label.

64. The kit of claim 53 or 63, wherein the detectable label is capable of being measured by a measurement of light scattering, optical absorbance, fluorescence, chemiluminescence, electrochemiluminescence (ECL), bioluminescence, phosphorescence, radioactivity, magnetic field, or combinations thereof.

65. The kit of claim 64, wherein the detectable label is an ECL label.

66. The kit of any one of claims 51 to 65, wherein the kit comprises a surface, and the surface comprises a particle.

67. The kit of any one of claims 51 to 65, wherein the kit comprises a surface, and the surface comprises a well of a multi-well plate.

68. The kit of claim 66 or 67, wherein the surface comprises an electrode.

69. The kit of claim 54 or any one of claims 56 to 68, further comprising a template oligonucleotide that is capable of binding to the nucleic acid probe.

70. The kit of any one of claims 55 to 68, further comprising a template oligonucleotide that is capable of binding to the first and/or second nucleic acid probes.

71. The kit of any one of claims 51 to 70, further comprising a polymerase, a ligase, a calibration reagent, a buffer, a co-reactant, a blocking agent, a diluent, a stabilizing agent, an assay consumable, an electrode, or a combination thereof.

72. A method of diagnosing Alzheimer's Disease (AD) in a subject comprising, when level of pTau181 in the subject are higher than 120% relative to a normalized concentration of pTau181 as measured by the method according to any one of claims 1 to 30, diagnosing the subject with AD.

73. A method of assessing risk for developing AD in a subject comprising:
when level of pTau181 and/or pTau217 in the subject are higher than 120% relative to a normalized concentration of pTau181 and/or pTau217 as measured by the method according to any one of claims 1 to 30, diagnosing the subject as having increased risk of developing AD; and
when level of pTau181 and/or pTau217 in the subject are lower than 50% relative to a normalized concentration of pTau 181 and/or pTau217 as measured by the method of any one of claims 1 to 30, diagnosing the subject as having decreased risk of developing AD

74. A method of preventing, reducing, or delaying AD in a subject comprising, when level of pTau181 and/or pTau217 in the subject are higher than 120% relative to a normalized concentration of pTau181 and/or pTau217 as measured by the method according to any one of claims 1 to 30, providing a regimen to the subject to prevent, reduce, or delay AD.

75. A method of diagnosing AD in a subject having increased likelihood of developing AD comprising, when level of pTau181 and/or pTau217 in the subject correspond to a positive likelihood ratio of greater than 5 when determined by the method of any one of claims 1 to 30, diagnosing the subject with AD.

76. A method of assessing risk of developing AD in a subject having increased likelihood of developing AD comprising:
when level of pTau181 and/or pTau217 in the subject provide a positive likelihood ratio of greater than 5 when determined by the method of any one of claims 1 to 30, determining the subject as having increased risk of developing AD; and
when level of pTau181 and/or pTau217 in the subject provide a negative likelihood ratio of 0.1 when determined by the method of any one of claims 1 to 30, determining the subject as having decreased risk of developing AD.

77. A method of preventing, reducing, or delaying AD in a subject having increased likelihood of developing AD comprising, when level of pTau181 and/or pTau217 in the subject correspond to a positive likelihood ratio of greater than 5 when determined by the method of any one of claims 1 to 30, providing a regimen to the subject to prevent, reduce, or delay AD.

78. A method of predicting cognitive decline in a subject with mild cognitive impairment (MCI) comprising:
when level of pTau181 and/or pTau217 in the subject are higher than 200% relative a normalized concentration of pTau181 and/or pTau217 as measured by the method of any one of claims 1 to 30, diagnosing the subject as having increased risk of cognitive decline; and
when level of pTau181 and/or pTau217 in the subject are lower than 75% relative to a normalized concentration of pTau181 and/or pTau217 as measured by the method of any one of claims 1 to 30, diagnosing the subject as having decreased risk of cognitive decline.

79. A method of preventing, reducing, or delaying cognitive decline in a subject with MCI comprising, when level of pTau181 and/or pTau217 in the subject are higher than 200% relative to a normalized concentration of pTau181 and/or pTau217 as measured by the method according to any one of claims 1 to 30, providing a regimen to the subject to prevent, reduce, or delay cognitive decline.

80. A method of predicting cognitive decline in a subject with MCI comprising:
when level of pTau181 and/or pTau217 in the subject provide a positive likelihood ratio of greater than 5 when determined by the method of any one of claims 1 to 30, determining the subject as having increased risk of cognitive decline; and
when level of pTau181 and/or pTau217 in the subject provide a negative likelihood ratio of about 0.1 to about 0.2 when determined by the method of any one of claims 1 to 30, determining the subject as having decreased risk of cognitive decline.

81. A method of preventing, reducing, or delaying cognitive decline in a subject with MCI comprising, when level of pTau181 and/or pTau217 in the subject correspond to a positive likelihood ratio of greater than 5 when determined by the method of any one of claims 1 to 30, providing a regimen to the subject to prevent, reduce, or delay cognitive decline.

82. The method of any one of claims 72 to 81, wherein the level of pTau181 and/or pTau217 in the subject is measured by a method according to any one of claims 1 to 30.

83. The method of any one of claims 1 to 30, wherein a lowest level of quantitation (LLOQ) for detecting pTau181 is less than 10 pg/mL, optionally less than 1 pg/mL.

84. The method of any one of claims 31 to 36, wherein an area-under-the-curve (AUC) value of a receiver-operating characteristic (ROC) curve for determining if the subject is likely or unlikely to experience cognitive decline is greater than about 0.7, optionally greater than 0.8.

85. The method of any of claims 1 to 30 or 82 to 84, wherein the level of pTau181 and/or pTau217 is measured using a MESO SCALE DIAGNOSTICS® ECL assay platform.

86. A method of detecting phosphorylated tau (pTau) in a sample, wherein the pTau is phosphorylated at amino acid position T217 (pTau217), comprising:
a) contacting the sample with: (i) a capture reagent that specifically binds pTau217; and (ii) a detection reagent that binds tau,
thereby forming a complex comprising the capture reagent, pTau217, and the detection reagent; and
b) detecting the complex, thereby detecting the pTau217.


SEQUENCE LISTING (1 sequence)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL